(19) United States
(12) Patent Application Publication
(10) Pub. No.: US 2020/0016195 A1
(43) Pub. Date: Jan. 16, 2020

(54) WATER-SOLUBLE NANOCERIA AND METHODS OF MAKING AND USING THE SAME

(71) Applicant: The University of North Carolina at Chapel Hill, Chapel Hill, NC (US)

(72) Inventors: Zongchao Han, Chapel Hill, NC (US); Rajendra Mitra, Carrboro, NC (US)

(21) Appl. No.: 16/485,934

(22) PCT Filed: Feb. 15, 2018
(86) PCT No.: PCT/US2018/018298

§ 371 Date: Aug. 14, 2019

Related U.S. Application Data
(60) Provisional application No. 62/459,294, filed on Feb. 15, 2017.

(51) Int. Cl. A61K 33/244; A61K 9/51; A61P 39/06; A61P 35/00

(52) U.S. Cl. CPC A61K 33/244; A61P 35/00; A61P 39/06; A61K 9/5161

(57) ABSTRACT

Water-soluble nanoceria are described herein along with methods of making and using the same. Water-soluble nanoceria described herein comprise a cerium oxide nanoparticle and glycol chitosan. In some embodiments, water-soluble nanoceria further comprise at least one therapeutic agent and/or targeting agent that may be attached to a surface of the water-soluble nanoceria.

### RELATED APPLICATION INFORMATION

This application claims the benefit of and priority to U.S. Provisional Application Ser. No. 62/459,294, filed Feb. 15, 2017, the disclosure of which is hereby incorporated by reference herein in its entirety.

### STATEMENT OF GOVERNMENT SUPPORT

This invention was made with government support under grant numbers EY026564 and EY024059 awarded by the National Institutes of Health. The government has certain rights in the invention.

### FIELD

The present invention relates to water-soluble nanoceria and to methods of making and using the same.

### BACKGROUND

Ceria oxide nanoparticles are named as an industrial vitamin. Ceria is a rare earth material that differs from main group elements and transition metals in the electronic configuration of 4f orbitals. The distinct electronic configuration of these orbitals generates unique physicochemical properties of this class of materials in catalysis, magnetic and other electronic properties. A ceria oxide nanoparticle, also known as nanoceria, adopts a face centered cubic structure (FCC). In addition to industrial applications for nanoceria as active components in catalysis etc., nanoceria have attracted attention as a multienzyme due to its enzyme mimicking properties like superoxide oxidase, catalase, and oxidase.

The most important feature of nanoceria lies in its dual state of oxidation. It remains either in +3 or +4 oxidation states. The relative ratio of +3 and +4 states depends on the particle size of nanoceria, where a decrease in particle size leads to dominant reduced state (+3) compared to the oxidized state (+4). Therefore, nanoceria plays a double role as oxidant and reductant based on reaction conditions and nanoparticle size. The shuffling between the two oxidative states is the main point of attention in nanoceria for its potential field of applications in therapeutics and diagnostics. Shuffling between the two oxidation states Ce⁺³/Ce⁺⁴ actually makes it capable of mimicking superoxide dismutase (SOD). SOD catalyzes the disproportionation (dismutation) of superoxide (O₂.) to H₂O₂ and O₂. It was observed that a higher ratio of Ce⁺³/Ce⁺⁴ leads to higher SOD mimicking activity.

Nanoceria also mimics the activity of catalase, a protective enzyme. Catalase degrades toxic H₂O₂. In the catalase like processes, Ce⁺⁴ is first reduced to Ce⁺³ by one molecule of H₂O₂ and generates O₂. In the second step, another H₂O₂ oxidizes Ce⁺³ back to Ce⁺⁴ and releases H₂O. Therefore, the SOD and catalase mimicking activity of nanoceria can degrade superoxide and hydroxyl radicals, the most destructive reactive oxygen species (ROS). The pH of the reaction condition greatly influences the catalytic activity of nanoceria. It has been observed that at physiological pH, nanoceria can mimic catalase activity, whereas in acidic pH it showed a disappearance of enzyme activity. Conversely, the SOD activity is independent of pH condition. Therefore, H₂O₂ degradation is affected by pH of the condition which remains unaffected for superoxide degradation. It has been demonstrated that with the addition of H₂O₂ to nanoceria (2-5 nm) with mixed valence states (Ce⁺³/Ce⁺⁴), colorless antioxidant Ce⁺³ was oxidized to yellow Ce⁺⁴ and upon storage in the dark for 30 days the yellow color reverted back to the colorless (Ce⁺³) nanoceria.

It has also been demonstrated that nanoceria can effectively scavenge the hydroxyl free radical. This auto-regenerative property of nanoceria makes it a potential antioxidant and therapeutically useful for neuroprotection. Nitric oxide radical (NO.) is another toxic ingredient which reacts with superoxide and forms peroxynitrite anion that is a more powerful toxic oxidant. Nanoceria can absorb and decompose NO. in industrial purpose and nanoceria have been shown to scavenge NO. radical with a lower ratio of Ce⁺³/Ce⁺⁴. Nanoceria can also direct Fenton-like reactions and can be utilized as a peroxidase mimic that catalyzes the reduction of peroxide.

Brain and central nervous systems are prone to oxidative damages due to low amounts of endogenous antioxidants, high levels of oxygen consumptions, and polyunsaturated fatty acids that lead to lipid peroxidations. However, an optimal antioxidant formulation yet remains to be established.

### SUMMARY OF THE INVENTION

A first aspect of the present invention is directed to a water-soluble nanoceria comprising a cerium oxide nanoparticle and glycol chitosan. A further aspect of the present invention is directed to a composition comprising the water-soluble nanoceria of the present invention.

Another aspect of the present invention is directed to a method of preparing a water-soluble nanoceria, the method comprising: combining a cerium salt and glycol chitosan to form a mixture; and adding ammonium hydroxide to the mixture to form a solution, thereby preparing the water-soluble nanoceria.

A further aspect of the present invention is directed to a method of treating an oxidative stress-related disorder, cancer and/or neurodegenerative disorder in a subject, the method comprising: administering a water-soluble nanoceria of the present invention or a composition of the present invention to the subject, thereby treating the oxidative stress-related disorder, cancer, and/or neurodegenerative disorder in the subject.

Another aspect of the present invention is directed to a method of decreasing reactive oxygen species in a subject, the method comprising: administering the water-soluble nanoceria of the present invention or a composition of the present invention to the subject, thereby decreasing reactive oxygen species in the subject.

The foregoing and other aspects of the present invention will now be described in more detail including other embodiments described herein.

### BRIEF DESCRIPTION OF THE DRAWINGS

FIGS. 1A-1E show information on glycol chitosan coated ceria nanoparticle (GCCNP) synthesis and their construct, morphology, size and surface charges. (A) A schematic illustration of the synthesis of GCCNPs and GCCNP constructs made of core ceria NPs within GC matrix. (B) Representative transmission electron microscopy (TEM) image of GCCNPs demonstrating similar shapes and sizes. Scale bar=1 μm. (C) Representative high resolution-TEM (HR-TEM) image of GCCNPs showing uniform shape and size of core ceria NPs (˜5 nm). In the inset, selected area electron diffraction (SAED) pattern reveals the fluorite lattice structure of core ceria NPs. Scale bar=5 nm. (D) Dynamic light scattering (DLS) of bared or uncoated ceria nanoparticles (BCNPs) and GCCNPs. Results expressed as mean±SEM were analyzed with the two-tailed unpaired Student's t-test, ***P<0.0001, n=5. (E) Zeta potential (surface charge) of BCNPs and GCCNPs. Results expressed as mean±SEM were analyzed with the two-tailed unpaired Student's t-test, ***P<0.0001, n=10.

FIGS. 2A-2F show characterizations of BCNPs and CNPs. (A) Raman spectra of BCNPs and GCCNPs. Peak appears around 455 cm⁻¹. (B) and (C) X-ray photoelectron spectroscopy (XPS) spectra of BCNPs and GCCNPs respectively. Peaks at 881 and 887 eV are related to Ce⁺⁴ and Ce⁺³. Satellite peak at 915 eV indicates the presence of Ce⁺⁴. (D) Thermogravimetric analysis (TGA) curves of BCNPs and GCCNPs. 71% weight loss happened due to successful GC coating on GCCNPs. (E) Optimization of fluorescence intensity of fluorescein at different concentrations. Arrow indicates 0.3 μM of Fluorescein sodium salt that was chosen from this experiment for further studies. (F) Antioxidant capacity of BCNPs and GCCNPs. Data was, analyzed using two-way ANOVA followed by Bonferroni's post hoc tests, ***P<0.001, n=4.

FIGS. 3A-3D show results from EPR studies and auto regenerative properties of BCNPs and GCCNPs. (A) Experimental X-Band (9.867 GHz) EPR spectra of aqueous solutions of (a) 47.62 mM DMPO, 0.238 mM Fe(II) sulfate, and 2.38 mM H₂O₂, (b) 47.62 mM DMPO, 0.238 mM Fe(II) sulfate, BCNPs (19.05 μM mM), and 2.38 mM H₂O₂; (c) 47.62 mM DMPO, 0.238 mM Fe(II) sulfate, GCCNPs (19.05 μM), and 2.38 mM H₂O₂; (d) 47.62 mM DMPO, 0.238 mM Fe(II) sulfate, GC (19.05 μM), and 2.38 mM H₂O₂; (e) 47.62 mM DMPO, BCNPs (19.05 μM), and 2.38 mM H₂O₂, signal amplitude is multiplied by 5; (f) 47.62 mM DMPO, GCCNPs (19.05 μM), and 2.38 mM H₂O₂ signal amplitude is multiplied by 5; (g) 47.62 mM DMPO and 2.38 mM H₂O₂, signal amplitude is multiplied by 5. The a-d spectra are averages of 5 scans, e-g spectra are averages of 100 scans measured at identical spectrometer settings. (B) Peak-to-peak amplitude of EPR signal from DMPO-OH. adducts at 294 K in the absence (a) and the presence of (b) 19.05 μM of BCNPs, (c) 19.05 μM of GCCNPs and (d) 19.05 μM GC. Time count starts from the moment of H₂O₂ addition during the final step of mixing. Dashed lines are best fits for the mono exponential decays. (C) Images of color changes of BCNPs (top) and GCCNPs (bottom) on addition of H₂O₂. These behaviors reflect the auto-regenerative nature of BCNPs and GCCNPs. (D) Plausible mechanism of free radical scavenging activity and auto-regenerative properties of BCNPs and GCCNPs.

FIGS. 4A-4F show quantitative assessments of in vitro cell viability, intracellular ROS scavenging, stimulating activities, as well as VEGF inhibition activity of GCCNPs in ARPE19 cells. (A) Quantitative cell viability of BCNPs and GCCNPs with ARPE-19 cell. 0.2 to 2.0 μM of NPs showed comparable cell viability with the untreated cells. At 4 μM of BCCNPs and GCCNPs showed viability 90%, whereas 10 μM of BCCNPs and GCCNPs demonstrated 81% and 74% of viability compared to the untreated control. Data was analyzed from four separate experiments. (B) The Intracellular ROS were detected with varying concentration of H₂O₂ (0.05-1.0 μM) and fixed DCFH-DA (50 μM). Yellow bar indicates the optimum 0.575 μM of H₂O₂ for further studies to avoid any possible toxicity with higher concentrations. Untreated=only media. Reagent only=only DCFH-DA treatments. Results presented as mean±SEM were analyzed with the two-tailed unpaired Student's t-test, ***P<0.0001, n=3. (C) Intracellular ROS scavenging activity of GCCNPs in ARPE19 cells. Results presented from three independent experiments as mean±SEM were analyzed with the two-tailed unpaired Student's t-test, ***P<0.0001. (D) Intracellular ROS was determined following the same method as above excluding H₂O₂. Data was analyzed using one-way ANOVA followed by Turkey's post hoc multiple comparison test, ***P<0.001, n=3. (E) Representative western blot analysis for H₂O₂ induced VEGF was performed with cell lysates collected from ARPE19 cells treated with GCCNPs (0.0, 0.5 and 1.0 μM). (F) Quantification showed that 1.0 ?AM GCCNP treatment significantly (**P=0.0011) reduced H₂O₂ induced intracellular VEGF expressions. Densitometric band analyses (mean±SEM) were presented from three independent experiments and were analyzed with the two-tailed unpaired Student's t-test, P<0.05 was taken as significant.

FIGS. 5A-5F show in vitro anti-angiogenic activity of GCCNPs. (A-F) Representative images (50× magnification) of tube-like structures of HUVEC cells were acquired at 8 hrs and treated with (A) 0 μM (B) 0.2 μM (C) 1.0 μM, (D) 5.0 μM of GCCNPs, as well as (E) 10 nM 2-Me (negative control). (F) Quantifications of number of tubular structures. Data shown are presented as mean±SEM (n=3) and analyzed using one-way ANOVA followed by Bonferroni's post hoc multiple comparison tests, **P<0.001, ***P<0.0001, and n=3.

FIGS. 6A-6D show delayed in vitro wound healing of HUVEC cells by GCCNP treatments. (A) Representative photographs (50× magnification) from scratch-wound assay were taken at 0 and 24 hrs after the generation of wounds in the monolayer cultures of HUVEC cells using a pipette tip. ECs at the edge of the scratch migrated towards the wound and closed it over time. The migrated distances were calculated. GCCNP (5 μM) treated cells inhibited almost 50% wound closure compared to the untreated control, whereas 10 nM of 2-Me treated cells could not close the wound, a negative control. (B) Quantification of EC migration, expressed as migrated distance. Migration was not detected in 2-Me (10 nM) treated cells, but was significantly delayed (=50%) on GCCNP (5 μM) treatments compared to the untreated control. Results represented as mean±SEM (n=3) by one-way ANOVA followed by Turkey's post hoc multiple comparison test. (C) Representative western blot analysis for H₂O₂ induced VEGF was performed with cell lysates collected from HUVEC cells after treatment with GCCNPs (0.0, 0.5, 1.0 and 5.0 μM). (D) Quantification of VEGF inhibition demonstrated that 5.0 μM GCCNP treatments significantly reduced (P=0.0165) H₂O₂ induced intracellular VEGF expressions compared to the untreated control. Densitometric band analyses were presented as mean±SEM from three independent experiments and analyzed with the two-tailed unpaired Student's t-test, P<0.05 was taken as significant.

FIGS. 7A-7B shows that GCCNPs inhibit neovascularization in image-guided laser-CNV mice model. FA and BF stand for fluorescein angiography and bright field respectively. (A) Representative fundus (FA and BF)/OCT photographs from intravitreal injections of saline (2 μl) treated eyes. Top panel corresponds to the fundus/OCT right after the laser injuries and just before injections, whereas bottom panel represents fundus/OCT at 14 days after laser injuries. (B) Representative fundus (FA and BF)/OCT photographs from intravitreal injections of GCCNP (2 μl from 0.4 μg/μl). Top panel corresponds to the fundus/OCT right after the laser injuries and just before injections, whereas bottom panel represents fundus/OCT at 14 days after laser injuries. Red colored arrows indicate the areas of laser damage.

FIGS. 8A-8E shows anti-angiogenic and laser-induced CNV targeting activities of GCCNPs. (A) Representative images from intravitreal injection of saline (2 μl) and GCCNP (2 μl from 0.4 μg/μl) laser-induced CNV RPE/choroid/scleral flat mounts. Scale bar=500 μm. (B) Quantitative measurements of laser-induced CNV areas (mm²). Results are presented as mean±SEM from saline (10 different eyes) and GCCNP (20 different eyes) treated eyes and analyzed with the two-tailed unpaired Student's t-test. (C) Representative image of western blot analyses of RPE/choroid/scleral tissues from intravitreal injections of saline (control, without GCCNPs, 2 μl), GCCNPs (2 μl from 0.4 μg/μl), and without laser-induced CNV and any injection as control. (D) Relative expressions of CXCR4 to β-actin, VEGF to β-actin, and 4-HNE to β-actin. Densitometric band analyses (mean±SEM) were carried out using one-way ANOVA followed by Turkey's post hoc multiple comparison test (n=4-6), *P<0.05 and **P<0.001. (E) GCCNPs were labeled with AF-SDP-488 ester and then injected (2 μl from 0.4 μg/μl) into intravitreal space of laser-induced CNV treated eye and then processed for RPE/choroid/scleral flat mount at 72 hrs. Representative photograph was presented that indicates the localization of GCCNPs-488 NPs into the laser-induced CNV lesions. Scale bar=500 μm.

FIG. 9 is a graph illustrating GCCNP size distribution.

FIGS. 10A-10B show graphs of reactive free radical generating capacity for GCCNPs at various concentrations in different cell lines. (A) GCCNPs increase the production of reactive oxygen species (ROS) at pH 6.5 but not at pH 7.4 in a dose dependent manner. (B) GCCNPs reduce cell viability in a pH-dependent manner in tumor cells but remain viable to normal ARPE19 cells up to 50 μM.

### DESCRIPTION OF EXAMPLE EMBODIMENTS

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as paving a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

It will be understood that when an element or layer is referred to as being “on”, “attached to”, “connected to”, “coupled to”, “coupled with” or “contacting” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. It will be appreciated by those of skill in the art that a structure referred to as being “directly on,” “directly connected to, or “directly coupled to” another structure may partially or completely cover one or more surfaces of the other structure. It will also be appreciated by those of skill in the art that references to a structure or feature that is disposed “adjacent” another structure or feature may have portions that overlap or underlie the adjacent structure or feature.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, the terms “increase”, “improve”, and “enhance” (and grammatical variants thereof) refer to an increase in the specified parameter of greater than about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300% or more.

As used herein, the terms “decrease”, “inhibit”, and “reduce” (and grammatical variants thereof) refer to a decrease in the specified parameter of about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%.

Provided herein are water-soluble nanoceria. The water-soluble nanoceria of the present invention may comprise a cerium oxide nanoparticle and glycol chitosan (GC). In some embodiments, the cerium oxide nanoparticle may be prepared from a cerium salt, such as, but not limited to, cerium chloride or cerium nitrate. In some embodiments, the cerium oxide nanoparticle may be prepared from a cerium chloride (e.g., cerium (III) chloride heptahydrate), cerium (III) nitrate hexahydrate, cerium (IV) sulphate, cerium oxalate, cerium carbonate, etc. Composition, size, and/or surface charge of the water-soluble nanoceria may be altered by using different weight ratios of GC to cerium salt (e.g., cerium chloride). In some embodiments, about 0.01 g to about 1.0 g (e.g., about 0.01 g, 0.02 g, 0.05 g, 0.1 g, 0.25 g, 0.3 g, 0.35 g, 0.4 g, 0.5 g, or 1.0 g) of GC may react with about 0.5 M to about 1 M (e.g., 0.67 M) of cerium salt (e.g., cerium chloride heptahydrate), the cerium salt optionally in a volume of about 0.1 mL to about 1 mL, to provide a cerium oxide nanoparticle.

In some embodiments, the cerium oxide nanoparticle may be present in an amount of about 10% to about 30% by weight of the water-soluble nanoceria and glycol chitosan may be present in an amount of about 60% to about 80% by weight of the water-soluble nanoceria. A water-soluble nanoceria of the present invention may comprise a cerium oxide nanoparticle in an amount of about 10%, 11%, 12%, 13%, 14%, 15%, 16%, 17%, 18%, 19%, 20%, 21%, 22%, 23%, 24%, 25%, 26%, 27%, 28%, 29%, or 30% by weight of the water-soluble nanoceria. A water-soluble nanoceria of the present invention may comprise glycol chitosan in an amount of about 60%, 61%, 62%, 63%, 64%, 65%, 66%, 67%, 68%, 69%, 70%, 71%, 72%, 73%, 74%, 75%, 76%, 77%, 78%, 79%, or 80% by weight of the water-soluble nanoceria. In some embodiments, the remaining weight percentage of the water-soluble nanoceria may be made up by water, such as, for example, in an amount of about 15% or less, such as, e.g., about 15%, 14%, 13%, 12%, 11%, 10%, 9%, 8%, 7%, 6%, 5%, 4%, 3%, 2%, 1%, or less.

The water-soluble nanoceria may include polyhydroxyl, ethylene glycol, and/or amine functional groups. In some embodiments, glycol chitosan (GC) coats at least a portion of an exterior surface of the cerium oxide nanoparticle such as, e.g., at least about 10%, 20%, 30%, 40%, 50%, 60%, 70%, 80%, 90%, 95%, or 100%. In some embodiments, GC coats substantially all of the outer surface of the cerium oxide nanoparticle. A GC coating on the surface of the cerium oxide nanoparticle may increase the water solubility and/or stability of the water-soluble nanoceria compared to nanoceria in the absence of GC, such as, for example, uncoated cerium oxide nanoparticles and/or a DMPO (5,5-dimethyl-1-pyrroline N-oxide) control.

Water-soluble nanoceria of the present invention may have an auto-regenerative property (i.e., may switch between two different oxidation states) under physiological conditions. In some embodiments, the water-soluble nanoceria may have a cerium (Ce) +3 state or a cerium +4 state. In a composition comprising a plurality of water-soluble nanoceria of the present invention, a portion of the plurality may have a cerium +3 state and another portion of the plurality may have a cerium +4 state. In some embodiments, the relative ratio of cerium +3 and cerium +4 states in the composition may depend on the particle size of water-soluble nanoceria. In some embodiments, a decrease in particle size may lead to a dominant reduced state (cerium +3) compared to the oxidized state (cerium +4). In some embodiments, the water-soluble nanoceria may have a surface charge close to neutrality, which may reduce non-specific interactions and/or self-aggregations. In some embodiments, the water-soluble nanoceria may have a positive surface charge.

In some embodiments, the water-soluble nanoceria of the present invention may be auto-regenerative and may scavenge and/or quench free radicals two or more (e.g., 2, 3, 4, 5, 6, or more) times during a given time period (e.g., 1, 2, 3, 4, 5, 6, 7, 8, 9, 10, or more hours, days, or weeks). The auto-regenerative property of the water-soluble nanceria may correspond to a change in oxidation from Ce³⁺ to Ce⁴⁺, then back to Ce³⁺ so that the nanoceria can once again scavenge and/or quench free radicals.

In some embodiments, at least a portion of the water-soluble nanoceria may be modified and/or functionalized with one or more different functionalities. For example, at least about 10%, 20%, 30%, 40%, 50%, 60%, 70%, 80%, 90%, 95%, or 100% of a surface of the water-soluble nanoceria may be modified and/or functionalized with one or more different functionalities. This may, for example, be due to the presence of and/or accomplished using primary amine groups on the surface of the water-soluble nanoceria. In some embodiments, water-soluble nanoceria may include chemically regenerative and/or natural antioxidant modalities in a common platform for scavenging pathological ROS.

In some embodiments, at least one therapeutic agent and/or targeting agent is attached to a surface of the water-soluble nanoceria. The at least one therapeutic agent and/or targeting agent may be covalently attached and/or linked to a surface of the water-soluble nanoceria. In some embodiments, the at least one therapeutic agent and/or targeting agent may be attached to the glycol chitosan on a surface of the ceria oxide nanoparticle, such as, e.g., attached to an amine functional group of the glycol chitosan. Example therapeutic agents may include, but are not limited to therapeutic proteins, drugs (e.g., active pharmaceutical ingredients), peptides, DNA, and/or the like. In some embodiments, the therapeutic agent is a chemotherapy drug. A targeting agent may be used to direct and/or attach the water-soluble nanoceria to a particle target (e.g., a cell or protein). Example targeting agents include, but are not limited to, antibodies, organic compounds, one portion of a binding pair (e.g., streptavidin and biotin), and/or the like. In some embodiments, the targeting agent (e.g., an antibody) is specific for an agent (e.g., a receptor) expressed on and/or by a cell of interest (e.g., a cancer cell). In some embodiments, water-soluble nanoceria comprising at least one therapeutic agent and at least one targeting agent may be able to specifically and/or selectively target tumor cells under tumor microenvironment conditions (e.g., acidic conditions), and the water-soluble nanoceria may not produce ROS or may produce ROS in a minimal amount (e.g., an amount that does not adversely affect cell viability by more than 5%, 10%, or 20% compared to cell viability in the absence of the water-soluble nanoceria) under normal physiological pH conditions, which may reduce the problem of developing drug resistance and/or insufficient specificity and/or selectivity. Water-soluble nanoceria of the present invention can be custom-built and/or functionalized to target different diseases and/or cells by binding a cell-specific targeting molecule and specific therapeutic agent to the surface of the nanoparticles.

A water-soluble nanoceria of the present invention may comprise a cerium oxide nanoparticle having a diameter, optionally an average diameter, in a range of about 2 nm to about 6 nm. In some embodiments, the cerium oxide nanoparticle may have a diameter of about 2, 2.5, 3, 3.5, 4, 4.5, 5, 5.5, or 6 nm. The cerium oxide nanoparticle and/or the coated core nanoparticle may be monodisperse and/or non-agglomerated. “Monodisperse” as used herein refers to cerium oxide nanoparticles having a uniform particle size, and, in some embodiments, having an average particle diameter ±5 nm (e.g., +5, 4, 3, 2, or 1 nm) as measured from electron micrographs. Glycol chitosan coated ceria nanoparticles may exhibit Z-average±160 nm as measured from dynamic light scattering, and/or having a polydispersity index≤0.2 as measured via dynamic light scattering. In some embodiments, water-soluble nanoceria of the present invention have an average particle diameter of about 5 nm or less.

Water-soluble nanoceria of the present invention may have a hydrodynamic diameter in a range of about 10 nm to about 1000 nm, such as, for example, about 50 nm to about 600 nm, about 100 nm to about 200 nm, or about 50 nm to about 200 nm. In some embodiments, the water-soluble nanoceria may have a hydrodynamic diameter of about 50, 100, 150, 200, 250, 300, 350, 400, 450, 500, 550, 600, 650, 700, 750, 800, 850, 900, 950, or about 1000 nm. In some embodiments, the water-soluble nanoceria may have a hydrodynamic diameter of less than 1000 nm or less than 600 nm. In some embodiments, the water-soluble nanoceria may have an average hydrodynamic diameter of about 50, 100, 150, 200, 250, 300, 350, 400, 450, 500, 550, 600, 650, 700, 750, 800, 850, 900, 950, or about 1000 nm. The water-soluble nanoceria may have a zeta potential in a range of about +10 to about +40. In some embodiments, the water-soluble nanoceria may have a zeta potential of about +10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In some embodiments, the water-soluble nanoceria may have a crystalline structure. The water-soluble nanoceria of the present invention may have a cubic fluorite structure.

In some embodiments, the water-soluble nanoceria of the present invention are ROS scavengers. The water-soluble nanoceria may quench free radicals (i.e., the water-soluble nanoceria may be a radical quencher). In some embodiments, the water-soluble nanoceria may mimic superoxide dismutase (SOD). In some embodiments, the water-soluble nanoceria may be an oxidizing agent, which may be used in a method of treating an oxidative stress-related and/or neurodegenerative disorder. The water-soluble nanoceria may act as an antioxidant and/or have a redox activity at a pH in a range of 6.5 or 7.0 to about 8 and/or at physiological pH, and/or the water-soluble nanoceria may have an intrinsic oxidase acidity at acidic pH and/or a pH less than 6.5.

Water-soluble nanoceria of the present invention may produce reactive oxygen species (ROS), optionally in a pH dependent and/or dose dependent manner. In some embodiments, the water-soluble nanoceria of the present invention have and/or provide an increased production of ROS at a pH in a range of about 6 to 6.5 or less than 7 compared to the production of ROS at a pH in a range of 7 to about 8, optionally in a dose dependent manner. In some embodiments, the water-soluble nanoceria of the present invention have and/or provide an increased production of ROS at a pH of about 6.5 compared to the production of ROS at a pH of about 7.4, optionally in a dose dependent manner.

In some embodiments, the water-soluble nanoceria of the present invention reduce cell viability in a pH dependent and/or dose dependent manner. In some embodiments, the water-soluble nanoceria may produce ROS in an amount sufficient to inhibit cell growth and/or cause cell death to a cell exposed to the ROS. In some embodiments, the water-soluble nanoceria of the present invention may preferentially kill and/or decrease cell viability of one or more cancer cells. In some embodiments, the water-soluble nanoceria of the present invention may preferentially kill and/or decrease cell viability of one or more cells having an intrinsic and/or extrinsic pH of less than 7.0 or 6.5. In some embodiments, the water-soluble nanoceria of the present invention, optionally when administered to a subject, may inhibit cell growth and/or kill a cancer cell and may not inhibit cell growth and/or kill a non-cancerous cell when present and/or exposed to the cells at the same concentration.

In some embodiments, water-soluble nanoceria of the present invention may be contacted and/or administered to a cell (e.g., a cancer cell), optionally present in a subject in an amount sufficient to inhibit cell growth and/or decrease cell viability (i.e., increase cell death in a population of cells) by at least about 20%, 25%, 30%, 35%, 40%, 45%, 50%, 55%, 60%, 65%, 70%, 75%, 80%, 85%, 90%, 95%, 98%, 99%, or 100% compared to the amount of cell growth and/or cell death in a control cell and/or population of cells not contacted and/or administered the water-soluble nanoceria.

The water-soluble nanoceria of the present invention may have an increased water solubility compared to nanoceria in the absence of GC, such as, for example, uncoated cerium oxide particles and/or a DMPO control. The water solubility may be increased by at least about 5% or more, such as, for example, about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300% or more compared to nanoceria in the absence of GC, such as, for example, uncoated cerium oxide particles and/or a DMPO control. In some embodiments, the water-soluble nanoceria of the present invention may have a water solubility in a range of about 0.0001 mg/ml to about 25 mg/ml, such as, for example, about 0.001 mg/ml to about 20 mg/ml, about 0.01 mg/ml to about 10 mg/ml, about 0.1 mg/ml to about 5 mg/ml, about 1 mg/ml to about 20 mg/ml, or about 0.01 mg/ml to about 1 mg/ml.

The water-soluble nanoceria of the present invention may have an increased residual time in a biological fluid (e.g., blood) compared to nanoceria in the absence of GC, such as, for example, uncoated cerium oxide particles and/or a DMPO control. The residual time in a biological fluid may be increased by at least about 5% or more, such as, for example, about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300% or more compared to nanoceria in the absence of GC, such as, for example, uncoated cerium oxide particles and/or a DMPO control.

The water-soluble nanoceria may be stable in an aqueous solution (e.g., water) for at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months or more, optionally at room temperature. “Stable” as used herein refers to substantially no or no degradation, substantially no or no aggregation, and/or substantially no or no change in solution color for a period of time. “Substantially” as used herein in reference to a result or value refers to a result or value that is within an error tolerance, has an average variance or change in value by less than 10%, and/or is not statistically significant. In some embodiments, the water-soluble nanoceria may be stable in an aqueous solution (e.g., water) at room temperature for at least about 1 year. In some embodiments, the water-soluble nanoceria may be stable in an aqueous solution (e.g., water) at room temperature for at least about 2 years.

In some embodiments, the water-soluble nanoceria may be stably stored as a lyophilized powder at a temperature in a range of about −25° C. to about 5° C. for at least about 6, 7, 8, 9, 10, 11, 12, 13, 14, 15, 16, 17, 18, 19, 20, 21, 22, 23, or 24 months or more with substantially no or no degradation, substantially no or no aggregation, and/or substantially no or no change in solution color upon reconstitution in an aqueous solution (e.g., water) compared to the properties of the water-soluble nanoceria in the aqueous solution at t=0.

In some embodiments, the water-soluble nanoceria may not change or may not substantially change in shape and/or size after storage at a temperature in a range of about −25° C. to about 5° C. for at least about 6, 7, 8, 9, 10, 11, 12, 13, 14, 15, 16, 17, 18, 19, 20, 21, 22, 23, or 24 months or more compared to the shape and/or size of the water-soluble nanoceria at t=0.

According to some embodiments of the present invention, a composition comprising a water-soluble nanoceria of the present invention may be provided. The composition may be an aqueous composition and thus may comprise water. The composition may be stable for at least about 6, 7, 8, 9, 10, 11, 12, 13, 14, 15, 16, 17, 18, 19, 20, 21, 22, 23, or 24 months or more.

In some embodiments, a method of preparing a water-soluble nanoceria of the present invention may be provided, the method comprising combining a cerium salt and glycol chitosan to form a mixture; and adding a base (e.g., ammonium hydroxide) to the mixture to form a solution, thereby preparing the water-soluble nanoceria. The cerium salt may be a cerium chloride, such as, for example, cerium (III) chloride heptahydrate. The method may be a green chemical method. In some embodiments, one or more steps of the method are carried out in an aqueous solution.

The cerium salt and glycol chitosan may be combined to form a mixture. In some embodiments, the cerium salt in an amount of about 0.1 M or 0.134 M to about 0.67 M, 0.7 M, or 1 M, optionally in an amount of about 0.1 mL to about 1 mL, is combined with about 0.05 g to about 0.25 g of glycol chitosan to form the mixture. The base (e.g., 28.0-30.0% ammonium hydroxide) may be added to the mixture in an amount of about 0.3 to about 1.5 mL. In some embodiments, the step of adding the base (e.g., ammonium hydroxide) to the mixture to form the solution may comprise adding water in an amount to increase the volume of the solution by about 10% to about 60%. In some embodiments, a cerium salt in an amount of about 0.01, 0.05, or 0.1 μmol to about 0.2, 0.5, or 1 μmol is combined with about 0.05 g to about 0.25 g of glycol chitosan or about 1 mL to about 10 mL of a 1% to about 5% w/v glycol chitosan solution to form the mixture.

In some embodiments, the step of combining the cerium salt and glycol chitosan may be performed at room temperature while stirring for a time period in a range of about 5 minutes to about 30 minutes. The method may further comprise centrifuging the solution, dialyzing the solution, neutralizing the solution to a pH in a range of about 7.2 to about 7.6, and/or lyophilizing the solution. In some embodiments, water-soluble nanoceria of the present invention are purified, solubilized, and/or stored in water, such as, for example, plain water (i.e., water with no added components (e.g., buffers), such as, e.g., purified water, deionized water, sterilized water, and/or distilled water).

In some embodiments, provided is a method of treating an oxidative stress-related disorder and/or neurodegenerative disorder in a subject, the method comprising administering a water-soluble nanoceria of the present invention and/or a composition of the present invention to the subject, thereby treating the oxidative stress-related disorder and/or neurodegenerative disorder in the subject. Exemplary oxidative stress-related disorders include, but are not limited to, age-related macular degeneration (AMD), diabetic retinopathy, retinopathy of prematurity, glaucoma, corneal disorders, dry eye syndrome, neurodegenerative diseases, Alzheimer's disease, Parkinson's disease, atherosclerosis, cancer, diabetes, rheumatoid arthritis, post-ischemic perfusion injury, myocardial infarction, cardiovascular diseases, chronic inflammation, stroke and septic shock, aging and other degenerative diseases. The subject may be any suitable subject, including, but not limited to, mammalian subjects, including both human subjects and animal subjects (e.g., dogs, cats, rabbits, cattle, horses, etc.), for diagnostic, therapeutic, research, and/or veterinary purposes. Subjects may be male or female and may be any age, including neonate, infant, juvenile, adolescent, adult, and geriatric subjects. In some embodiments, the subject is need of a method of the present invention.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with a disease or disorder (e.g., an oxidative stress-related disorder and/or neurodegenerative disorder) is achieved and/or there is a delay in the progression of the disease or disorder. In some embodiments, the severity of an oxidative stress-related disorder and/or neurodegenerative disorder may be reduced in a subject compared to the severity of the oxidative stress-related disorder and/or neurodegenerative disorder in the absence of a method of the present invention. In some embodiments, a method of the present invention treats an oxidative stress-related disorder, such as, but not limited to, vascular disease, diabetes mellitus, and/or cancer in a subject, and/or a neurodegenerative disorder in a subject, such as, but not limited to, age-related macular degeneration, choroidal neovascularization, retinal neovascularization, diabetic retinopathy, glaucoma, retinitis pigmentosa, sickle cell retinopathy cataract, dry eye syndrome, retinal detachment, retinopathy of prematurity, neovascular glaucoma, macular edema, ocular oncology, ocular inflammations, corneal disorders, conjunctivitis, cardiovascular disease, cancer, and/or Alzheimer's disease. In some embodiments, provided is a method of treating cancer and/or increasing cancer cell death in a subject, the method comprising administering a water-soluble nanoceria of the present invention and/or a composition of the present invention to the subject, thereby treating cancer in the subject and/or increasing cancer cell death in the subject compared to cancer cell death in the absence of a method of the present invention.

Provided in some embodiments is a method of decreasing reactive oxygen species in a subject, the method comprising administering a water-soluble nanoceria of the present invention and/or a composition of the present invention to the subject, thereby decreasing reactive oxygen species in the subject.

In some embodiments, water-soluble nanoceria of the present invention and/or a composition of the present invention is administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount of a composition of the present invention may be administered and may include administering a treatment effective amount of the water-soluble nanoceria.

In some embodiments, a water-soluble nanoceria of the present invention and/or a composition of the present invention is administered in therapeutically effective amount. As used herein, the term “therapeutically effective amount” refers to an amount of water-soluble nanoceria of the present invention and/or composition of the present invention that elicits a therapeutically useful response in a subject. Those skilled in the art will appreciate that the therapeutic effects need not be complete or curative, as long as some benefit is provided to the subject.

Water-soluble nanoceria of the present invention and/or a composition comprising the same may be administered using any suitable method. In some embodiments, parenteral administration may be used. “Parenteral administration” as used herein includes, but is not limited to, intravenous, subcutaneous, intramuscular, intraperitoneal, intraarterial, intraosseous, intrathecal or intraventricular administration, e.g., through injection or infusion. In some embodiments, the water-soluble nanoceria and/or composition may be injected into a subject (e.g., an intravitreal injection). In some embodiments, after intraocular injection, the water-soluble nanoceria may not be distributed to the subject's blood (due to the physical barriers of the eyes).

In some embodiments, administration of water-soluble nanoceria of the present invention and/or a composition of the present invention may inhibit and/or decrease the growth of neovascularization in a subject. In some embodiments, the water-soluble nanoceria of the present invention and/or a composition of the present invention may be non-toxic to the subject and/or have low or no cytotoxicity. In some embodiments, a composition of the present invention may be administered to a subject in an amount of about 1 μL or 5 μL to about 10 μL, 15 μL, or 20 μL with the composition having a concentration of water-soluble nanoceria in a range of about 0.1 μg of water-soluble nanoceria per μl of the composition to about 10 μg of water-soluble nanoceria per μl of the composition. In some embodiments, the composition may have a concentration of water-soluble nanoceria in an amount of about 0.1, 0.5, 1, 1.5, 2, 2.5, 3, 3.5, 4, 4.5, 5, 5.5, 6, 6.5, 7, 7.5, 8, 8.5, 9, 9.5, or 10 μg of water-soluble nanoceria per μl of the composition.

### EXAMPLES

### Example 1

### Synthesis and Physicochemical Characterizations of Ceria Nanoparticles

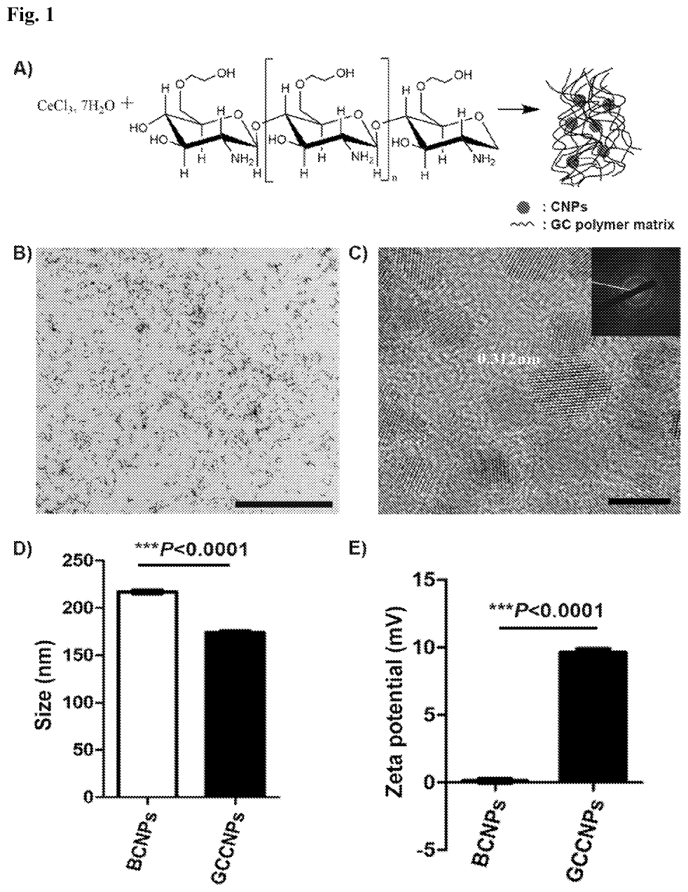

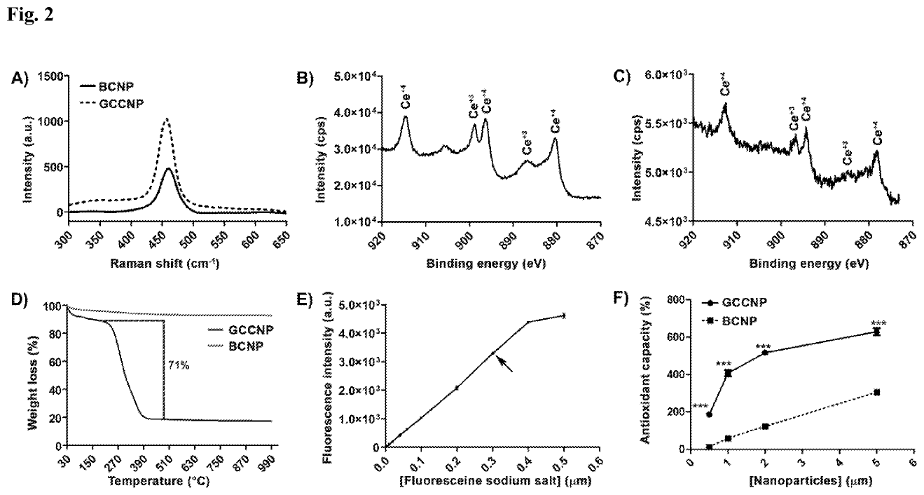

In this study, ceria nanoparticles were prepared using a NH₄OH precipitation method and coated with GC (FIG. 1A). The 0.25 g of glycol chitosan (Sigma-Aldrich, USA) was first dissolved in 15 ml of warm water (˜70° C.) under constant stirring for 1-2 hrs. in glass vial. The glycol chitosan solution was then stirred again at room temperature (20° C.) for another 1 hr. 0.67M (0.25 g in 1 ml of water) cerium (III) chloride heptahydrate was added dropwise to the clear glycol chitosan solution under constant stirring at 5000 rpm and room temperature to form a reaction mixture A. After 15-20 minutes of constant stirring, 1.5 ml of 28.0-30.0% ammonium hydroxide (Sigma-Aldrich, USA) was dropwise added to the reaction mixture A to form reaction mixture B, which was left stirring for another 12 hrs at room temperature. The color of the reaction mixture B turned to light yellowish on next day. This mixture B was then stirred under vacuum to remove the ammonium smell. Mixture B was then centrifuged at 4000 rpm at room temperature for 20 minutes. Clear supernatant of mixture B was carefully separated from the little amount of debris and then dialyzed against nanopure water using dialysis membrane (12-14 kDa molecular weight cut off) for 3 days with 10 times water changes. After complete dialysis, pH of the clear yellowish solution was neutralized with glacial acetic acid to pH 7.4. This final glycol chitosan coated ceria nanoparticle solution was lyophilized and stored at −20° C. for future purpose. HR-TEM images (FIGS. 1B and C) revealed that the GC coated CNPs were 3-5 nm in diameter. The crystalline nature (lattice constant 0.312 nm) and cubic fluorite structure of CNPs were confirmed by the selected area electron diffraction (SAED) pattern in the inset of FIG. 1C. The average hydrodynamic diameter (D_{H}) of GC coated ceria nanoparticles (GCCNPs) and bared or uncoated CNPs (BCNPs) were determined from DLS studies as 174±1.37 nm and 217±2.14 nm respectively, as demonstrated in the FIG. 1D. The higher D_{H} value of the BCNPs is due to the agglomeration or aggregation into clusters which justifies their lower solubility in water. The zeta potential of GCCNPs (9.6±0.26 mV, FIG. 1E) was also significantly (***P<0.0001) higher than the neutral BCCNPs (0 mV). The neutral charge and large size of BCNPs might be responsible for its lower stability in water, whereas the positive surface charge, ethylene glycol and free hydroxyl groups on the surface of GCCNPs ensure higher aqueous solubility of GCCNPs. The pH of the GCCNPs solution was found to be 7.4 and these NPs were stable throughout the year in water even at room temperature and without changing color of the solution. The most important advantage of GCCNPs is that it can be preserved for a long time in lyophilized condition. The symmetrical stretching band of C—O at 458 cm⁻¹ (FIG. 2A) is strongly correlated with Raman active mode (F2g) of cubic fluorite structure of CNPs. This Raman spectra of cerium oxide nanoparticles is strongly correlated with the previous literature²⁹ and confirms the cubic fluorite structure of both BCNPs and GCCNPs. The faint yellow color of the GCCNPs indicated the presence of both Ce⁺³/⁺⁴ oxide mixtures. To verify that, we carried out XPS analysis with BCNPs and GCCNPs as shown in FIGS. 2B and 2C. The XPS analysis of 3d peaks across the cerium oxide surface demonstrated that GCCNPs had the same Ce⁺³/Ce⁺⁴ (oxidation/reduction) pattern as BCNPs, and thus confirms the presence of both oxides in GCCNPs, which was not changed by coating. This observation also illustrates the color of GCCNPs as light yellow due to the mixture of Ce⁺³ (colorless) and Ce⁺⁴ (yellowish) oxides. These compositions ensure the redox cycling between Ce⁺³ and Ce⁺⁴ in our nano-formulation and explores GCCNPs as an auto regenerative and robust antioxidant system. We have also carried out a straight forward TGA analysis to quantitatively determine the coverage of GC on the ceria nanoparticle surface. FIG. 2D showed TGA curve of BCNPs and GCCNPs in the range of 30-990° C. and under inert nitrogen atmosphere. The TGA curve of GCCNPs showed a small weight loss under 110° C. due to desorption of adsorbed water (moisture, 9%) followed by a continuous weight loss (91-20%) within the range of 110-400° C. due to the decomposition of GC coated on the surface of naked ceria nanoparticles. The 20% weight left over was due to the inorganic cerium content into the GCCNPs complex. This result demonstrated that our present methodology was able to significantly and uniformly coat the CNP surface by GC polymer (71% weight loss).

### GC Coating Improved the Antioxidant Capacity of Uncoated Ceria Nanoparticles as Determined by ORAC Assay

Herein, we performed ORAC that is widely used to measure the antioxidant capacity of nutraceuticals, pharmaceuticals, and foods. In this assay, a fluorescent probe (fluorescein sodium salt) is used and the changes in fluorescence of that probe in presence or absence of GCCNPs and radical initiator (AAPH) was determined, throughout a time period, to calculate the area under the fluorescence decay curve (AUC). An initial attempt was achieved to determine an optimum concentration (0.3 μM) of the fluorescent probe (FIG. 2E) to accomplish further ORAC assays. The assay results (FIG. 2F) revealed that the antioxidant capacity of GCCNPs was significantly (***P<0.0001) higher than the uncoated BCNPs within our experimental dose range of 0.5-5.0 μM. This high antioxidant capacity reflected the classical ability of GCCNPs to quench free radicals at a significantly higher rate than that of the uncoated BCNPs.

### EPR Analysis Revealed GC Coating Improved the Hydroxyl Free Radical Scavenging Activity of GCCNPs Compared to the BCNPs

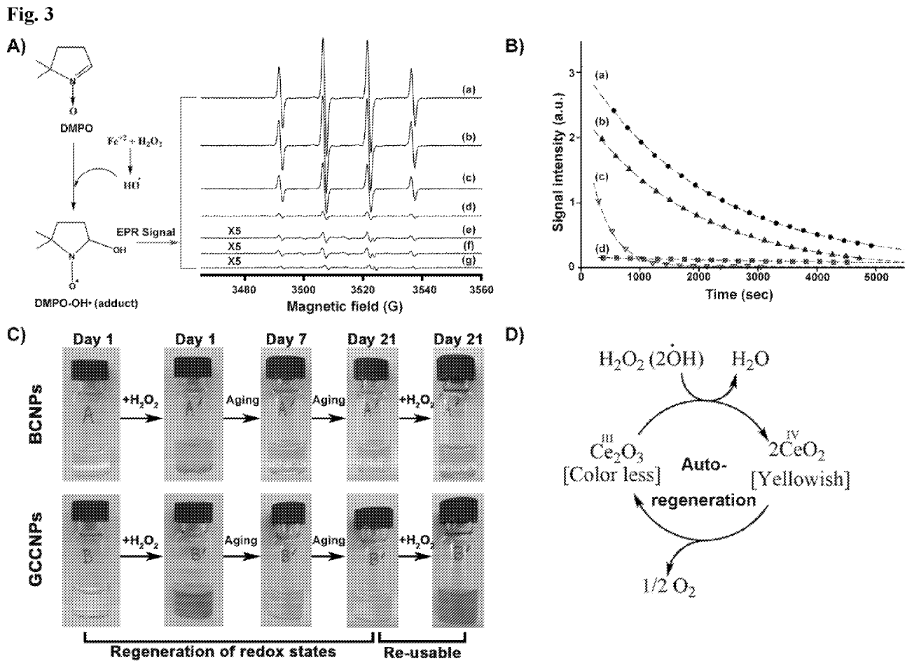

EPR spin-trapping technique is a powerful and direct analytical method for quantification of short lived small radicals that is commonly employed for characterization of the catalytic production of reactive oxygen species, including studies of nanomaterials³⁰. In our work, we focused on monitoring the production of hydroxyl radicals that are considered to be the most harmful among all the ROS. The radical production has been assessed by trapping short-lived radicals with a diamagnetic compound, 5,5-dimethyl-1-pyrroline N-oxide (DMPO) and the nature of the trapped radical was identified from the magnetic parameters of the observed EPR spectra. Hydroxyl radicals were generated by mixing solutions of iron sulfate, DMPO, and the sample of interest (or water in the control experiment) followed by an addition of hydrogen peroxide to start the reaction. In these experiments, we observed a four-line EPR signal with a 1:2:2:1 peak-to-peak intensity pattern and the isotropic hyperfine coupling constants (AN=AH≈14.9 G) that are characteristic of the DMPO-OH adduct (FIG. 3A). FIG. 3B shows time decay of the EPR intensity for the spin-adduct signal generated at the absence and in the presence of the GC, BCNPs and GCCNPs. The time scale starts at the moment of addition of hydrogen peroxide to the mixtures. In a control experiment, the EPR signal intensity was well approximated by an exponential decay, with the effective half-life of the signal (assuming first-order process) t_{1/2}=26 min. This result was consistent with the half-life time of DMPO—OH. adduct reported by Villamena and co-authors³¹ in absence of iron ions that are known to accelerate the adduct decay³². When the hydroxyl radicals were generated and trapped in the presence of the GC, a drastic reduction of the EPR signal was observed. Although the intensity of the EPR signal generated in the presence of the GC (FIG. 3A, d) was measurably higher than the background signal detected from DMPO and hydrogen peroxide (FIG. 3A, a), the presence of GC in the reaction mixture effectively blocks trapping of the hydroxyl radicals by DMPO, indicating, for the first time, that GC scavenges the radicals more efficiently than DMPO. In the presence of BCNPs (FIG. 3A, b) the initial EPR signal intensity dropped by approximately 20%, indicating that although BCNPs possess some radical scavenging properties, it was not as effective in the reaction with hydroxyl radicals as DMPO. Spin trapping in the presence of GCCNPs (FIG. 3A, c) resulted in a significant decrease of the initial signal intensity, by an approximate factor of 3 as compared to the control experiment (FIG. 3A, a). This observation, for the first time, suggests that GCCNPs is much more effective than DMPO in the reaction with hydroxyl radicals and much more effective as a radical quencher than BCNPs. In addition to the observed drop in the initial intensity of the spin-adduct signal, the effective rate of the spin-adduct decay increased by a factor of 5 (FIG. 3B), supporting the conclusion that GCCNPs showed an effective quenching of hydroxyl radicals.

In the next set of experiments, we have tested the ability of GC, BCNPs and GCCNPs to catalyze generation of hydroxyl radicals from hydrogen peroxide. In these measurements, hydrogen peroxide was added to a mixture of DMPO solution and BCNPs (FIG. 3A, e) or GCCNPs (FIG. 3A, f) and the EPR signal was monitored. The intensities of the detected four-line EPR signals (FIG. 3A, e and f) were similar to that detected upon addition of the DMPO to hydrogen peroxide (FIG. 3A, g).

### Auto-Regeneration, Reusable Properties of BCNPs and GCCNPs

To illustrate the auto-regenerative properties of BCNP and GCCNP, we added 0.1 (M) H₂O₂ to 20 mM stocks of those nanoparticles (FIG. 3C), which quickly changed the color of the samples to dark yellow (day 1). On day 21, the color of samples reverted back to colorless. Therefore, these result confirmed that the nanoparticles are auto-regenerative in nature. To demonstrate the reusability of these nanoparticles, we repeated the oxidation process with 0.1 (M) H₂O₂ additions on 21 day with same samples. Excitingly, we observed that the nanoparticles were reusable and had the abilities to continue scavenge free radicals. In these experiments, BCNPs remained insoluble whereas GCCNPs demonstrated a clear solution form in water (FIG. 3C). Hence, this study reflected the unique auto-regenerative, reusable and water soluble properties of novel GCCNPs. The plausible mechanism of the redox reaction is presented in the FIG. 3D.

### GCCNPs Demonstrated Biocompatibility Towards ARPE19 Cells

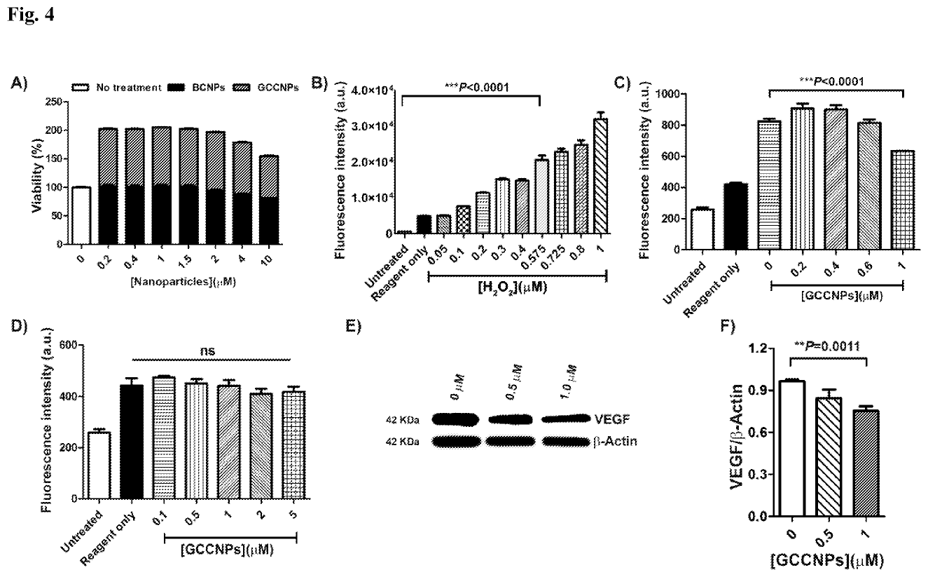

Initial experiments were performed to evaluate the cytotoxicity of the GCCNPs for human RPE cells (ARPE19) at 24 hrs of NP treatments. Under the experimental condition, up to 2 μM of GCCNPs barely affected the viability of the cells and remained comparable with that of BCNPs as shown in FIG. 4A. At 4 μM of GCCNPs, 90% of cells could survive compared to the untreated control, whereas for BCNPs survival went down to 88%. At 10 μM of GCCNPs and BCCNPs, 74% and 81% of cell viabilities were observed respectively. Thus, treatment of ARPE19 cells with GCCNPs and BCNPs were dose dependent and decreased cell survival with increase in the concentration of nanoparticles compared to that of the untreated control. Therefore, we have chosen much lower concentration of GCCNP (0.2-5 μM) for our further in vitro and in vivo experiments to ensure its minimum toxicity.

### GCCNPs Scavenged Intracellular ROS

Excessive production of ROS plays a decisive role in the pathogenesis of AMD. Daily ingestion of oxidized photoreceptor sheds leads to high metabolic rate of neuronal retina and high rate of energy consumptions, which make them susceptible to any physiological imbalances. In the aged retina, antioxidant defense system is also vulnerable to imbalances and results in the accumulation of ROS in the RPE cells, which further causes oxidative stress³³. To determine scavenging effect of GCCNPs on the intracellular production of ROS with in vitro model, we evaluated the generation of ROS in ARPE19 cells, a human RPE cell line. To investigate whether the H₂O₂ can induce ROS production inside the ARPE19 cells, we first attempted to optimize the intracellular ROS by different doses of H₂O₂ (0.05-1.0 μM). The intracellular ROS levels were determined by the cell permeable and oxidation sensitive DCFH-DA. The ROS oxidation of this dye generates highly fluorescent intracellular DCF. FIG. 4B demonstrated the mean fluorescence of DCF (i.e. intracellular ROS generation) increased gradually and significantly up to 1.0 μM of H₂O₂ compared to the untreated and DCFH-DA (reagent) treated controls. The significant amount of DCF fluorescence increased from 0.05 μM to 0.575 μM of H₂O₂ (10-fold change) was sufficient to choose 0.575 μM as a standard concentration of H₂O₂ for further studies.

To assess the antioxidant activity of GCCNPs, the intracellular ROS scavenging activity was measured against exogenous H₂O₂. We cultured ARPE19 cells in 96 well plates with cell density of 10,000 cells/well. The cells were treated with GCCNPs (0.2-1.0 μM) for 24 hrs and then incubated with DCFH-DA followed by activation with H₂O₂ to induce ROS. As shown in FIG. 4C, there is a significant decrease in DCF fluorescence, an ROS indicator, at 1.0 μM of GCCNPs compared to the H₂O₂ activated control (i.e. without GCCNPs) which clearly demonstrated a remarkable suppressive effect of 1.0 μM GCCNPs (***P<0.0001) on intracellular ROS generation compared to untreated control. These results suggest that 1.0 μM of GCCNPs preferentially scavenged the intracellular ROS and attenuated the conversion of DCFH to DCF within APRE19 cells.

We next examined the ROS stimulating activity of the GCCNPs, we carried out the same assay as above with GCCNPs but in absence of H₂O₂ (FIG. 4D). The results revealed that the GCCNPs (0.1-5 μM) didn't create any DCF fluorescence and remained comparable to fluorescence of DCFH-DA (reagent only) treated cells. These results revealed that the GCCNP doesn't induce any intracellular ROS.

GCCNPs Suppressed H₂O₂ Induced Intracellular VEGF in ARPE19 Cells

VEGF mediated CNV is the hallmark of wet AMD. The oxidative stress in the retina can lead to the accumulations of oxidized debris and pathological ROS that cause dysfunction of RPE cells and choroidal angiogenesis, which promote wet AMD. Therefore, protection of RPE cells from ROS induced oxidative stress is an unmet need to reduce the pathogenesis of wet AMD. To address this issue, we examined the effect of antioxidant (GCCNPs) on the H₂O₂-induced oxidative stress in ARPE19 cells, a classical in vitro model for human AMD^{33a}. We cultured ARPE19 cells (1.5×10⁵ cells/well) for 24 hours with and without GCCNPs (0.5 and 1.0 μM) followed by incubation with H₂O₂ (0.575 mM) for another 24 hrs under serum free culture medium. The intracellular VEGF was detected by western blot analysis (FIG. 4E). The result showed that 1.0 μM of GCCNPs significantly (**P=0.0011) down regulated intracellular VEGF expression (FIG. 4F).

### GCCNPs Inhibited Capillary Tube Formations and Migrations of HUVEC

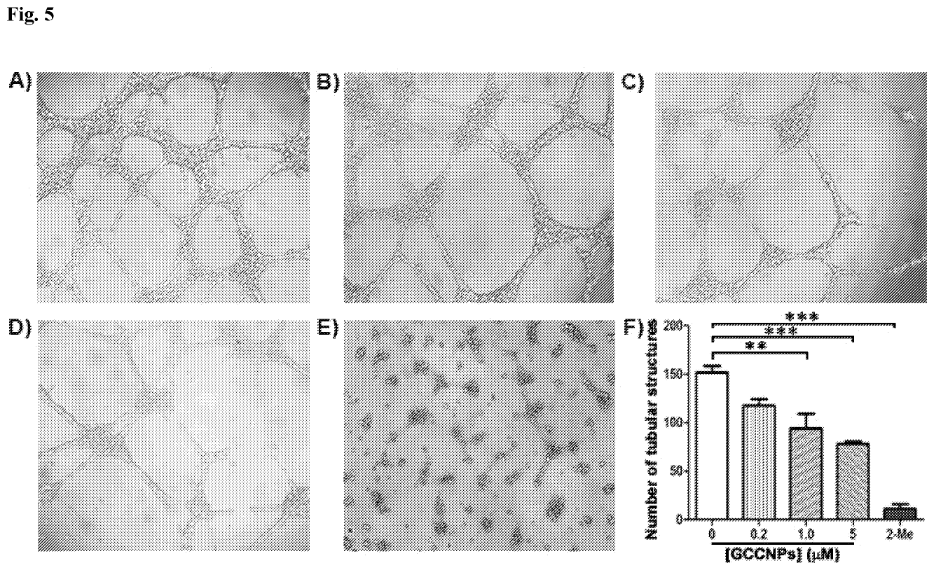

The formation of tube like structures of ECs through the base membrane matrix is a robust in vitro tool to screen materials in inhibiting or promoting angiogenesis. To test our hypothesis that GCCNPs inhibit angiogenesis, as mentioned in earlier studies with ceria nanoparticles¹¹, we performed tube formation assay with different doses of GCCNPs. Capillary tube formations were monitored and quantitatively analyzed at 8 hrs (FIG. 5). The GCCNPs (0.2-5 μM), to avoid excessive toxicity, were incubated with HUVEC cells seeded (25,000 cells/well) in Matrigel (FIG. 5B-D), which revealed that the tubular structures were gradually reduced in a dose dependent manner (FIG. 5B-D) compared to the untreated control (FIG. 5A). A quantitative analysis showed that 1.0 and 5.0 μM of GCCNPs significantly (**P<0.001 and ***P<0.0001 respectively) reduced the growth of tube-like structure network (FIG. 5F) compared to the untreated control (FIG. 5F). The treatment of HUVECs with 2 -Me (10 nM, 2-methoxyestradiol, a metabolite of estradiol—17 beta), an angiogenic inhibitor of ECs proliferation and angiogenesis, exhibited a direct inhibition of tube inductions (FIGS. 5E and 5F).

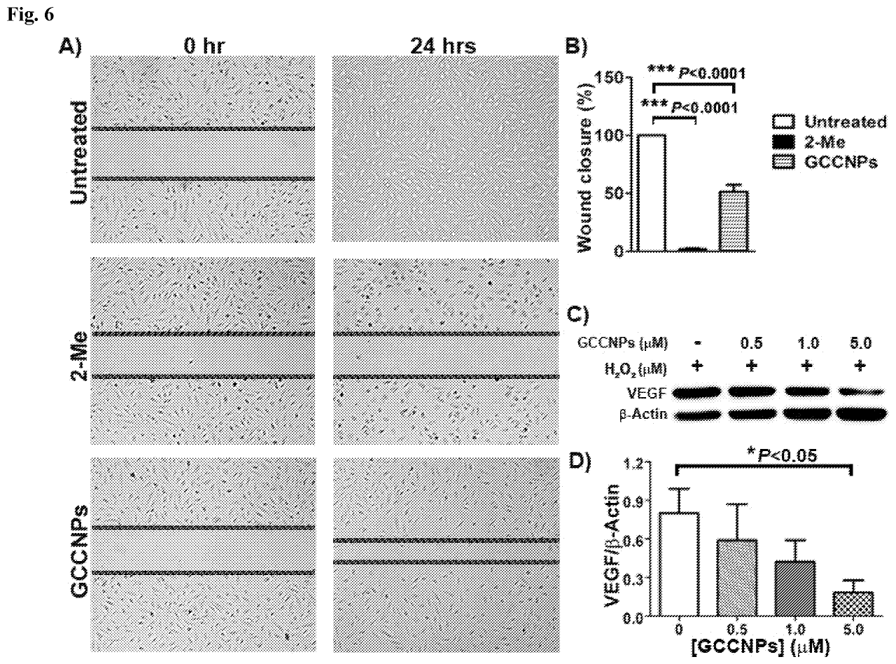

The endothelial cell migration is a crucial step for tubule formation and thus we evaluated the migratory response of ECs to GCCNPs (FIGS. 6A and 6B) after 24 hrs of incubation. To this end, we performed a scratch-wound assay on HUVEC monolayers (FIG. 6A). Consistent with the inhibition of tube like structures of ECs, GCCNP (5 μM) treated cells also attenuated wound closures (˜50% closure, ***P<0.0001) compared to the untreated control (FIGS. 6A and 6B). The treatment of HUVECs with 2-Me (10 nM) led to complete inhibition (FIGS. 6A and 6B) of wound closures and was consistent with its tube formation assay (FIGS. 5E and 5F). Our results are consistent with the earlier study where nanoceria and heparin conjugated ceria NPs (heparin-nanoceria) demonstrated a significant amount of inhibition of EC growth¹⁷.

GCCNPs downregulated H₂O₂ induced intracellular VEGF in HUVEC cells

We were excited to see the effect of novel GCCNPs formulation in regulating the VEGF expression induced by the H₂O₂ inside the HUVEC cells. It is observed that H₂O₂ can stimulate the VEGF induction in cultured ECs and might be a good model to evaluate the anti-angiogenic activity of GCCNPs against ROS induced intracellular VEGF expression³⁴. The HUVEC (2.5×10⁵ cells/well) cells were initially exposed to different concentrations of GCCNPs (0.5, 1.0 and 5.0 μM) in a 6-well plate with untreated control. After 24 hrs of GCCNPs treatments, the cells were further treated with H₂O₂ (0.25 mM) for another 6 hrs at 37° C. and 5% CO₂ incubator. Here, the protein analysis showed that the cellular VEGF expressions were also down regulated with different concentration of GCCNPs (FIG. 6C). A quantitative analysis showed that 5.0 μM of GCCNPs significantly inhibit (*P<0.05) the intracellular VEGF expression compared with the untreated control (FIG. 6D).

### Intravitreal Injection of GCCNPs Attenuated Laser-Induced CNV

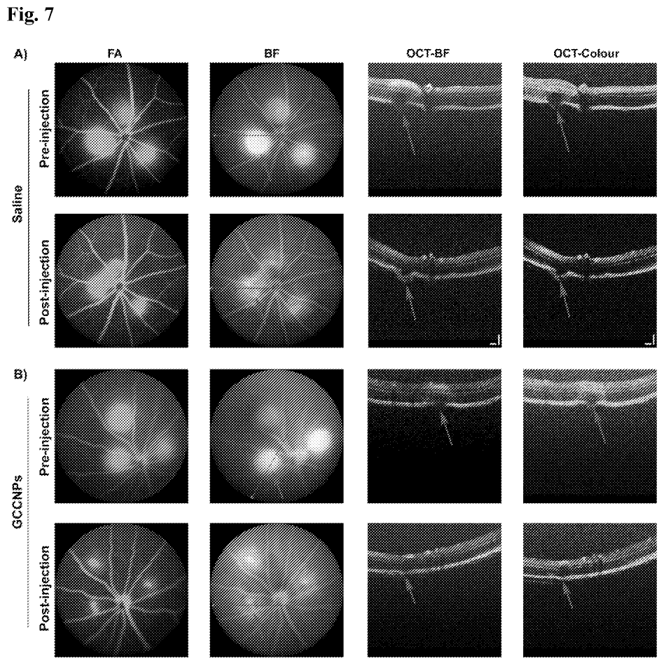

The in vitro analyses demonstrated that the GCCNPs are robust antioxidants that can scavenge ROS and also inhibit angiogenesis by down regulating pro-angiogenic VEGF. Therefore, we were excited to explore if GCCNPs play any significant role in the inhibition of neovascularization in a laser-induced CNV mouse model, a classical and extensively used standard animal model of wet AMD. In wet AMD, the growth of new vessels typically branches from the pre-existing one and leads to hemorrhages due to the increase in microvascular permeability. We examined the fundus fluorescein angiography (FA) and optical coherence tomography (OCT) immediately after the laser insults (i.e. before injection) to confirm the success of the laser burns. Laser injuries disrupt Br and promote the generation of new vessels from choroid across the RPE to the subretinal space (top panels of FIG. 7). A good correlation between FA and OCT can help us to minutely monitor the failure and success of laser-induced CNV lesions and progress over treatments. Immediately after the CNV lesion FA and OCT analyses, we performed a single intravitreal injection of saline (2 μl/eye) and/or GCCNPs (2 μl/eye) to each laser-induced mouse eye. Only the successful laser injuries were included in our studies. The final FA and OCT images were collected to determine the effect of saline and GCCNPs on the CNV lesions at 14 days post laser photocoagulation, a time point that reaches the maximum damage range³⁵. The OCT images acquired with FA confirms the rupture of BM right after the laser injuries in the eyes (indicated by the arrow in FIGS. 7A and 7B). The proliferation of new blood vessels from the laser induced injuries were detected by FA and taken into account for the quantitative assessment of laser-induced CNV. GCCNP injections (2 μl from 0.4 μM stock) revealed an inhibition of FA or the reduction in laser-induced CNV damage area compared to that of the saline injected controls (FIGS. 7A and 7B) at 14 days post laser treatments. We followed these experiments with the 2D cross sectional OCT scans (FIGS. 7A and 7B). OCT analyses exhibited a reduction in the thickness of laser-induced CNV injuries compared to saline injected controls.

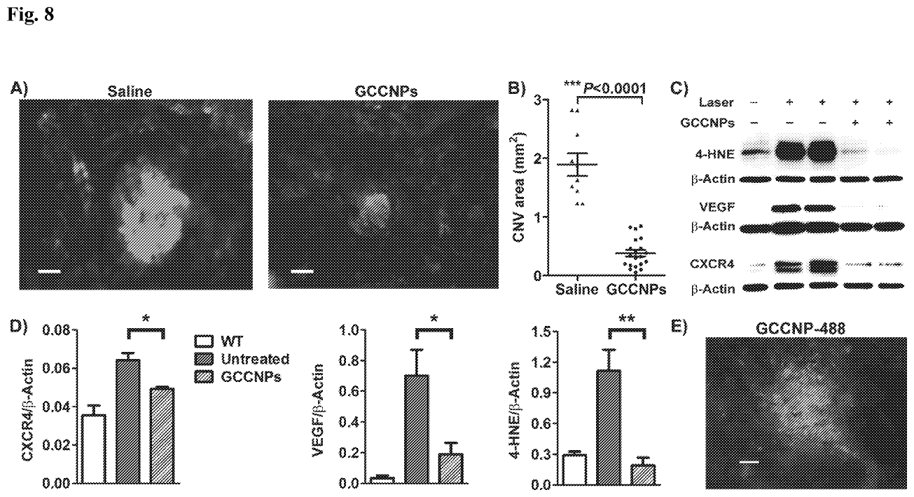

To reassure the FA/OCT results and to quantitatively analyze the results, mice were sacrificed and choroidal flat mounts were prepared and stained with Alexa Fluor-488 conjugated Griffonia simplicifolia isolectin-IB4 (GS-IB4, Thermo fisher, Cat. No. 121411), an endothelial cell specific marker. As shown in FIG. 8A, the mean CNV area of GCCNP treated eyes was significantly reduced compared to the saline treated controls. A quantitative analyses demonstrated that GCCNPs treatments significantly regress the CNV (***P<0.0001) compared to saline injected control (FIG. 8B).

### GCCNPs Reduced VEGF, Pro-Inflammatory Chemokine (CXCR4) and Oxidative Stress 4-HNE Adducts

Retina, with the abundance of large amounts of PUFAs (lipids), is highly prone to ROS mediated oxidations. The lipid peroxidation in the retina generates highly toxic aldehyde 4-hydroxynonenal (4-HNE) that further covalently conjugates with different amino acids of protein to form stable protein adduct (4-HNE adduct), a biomarker of AMD, which in turn promotes the generation VEGF^{28a}. Increased VEGF promotes the pathogenesis of CNV in laser-induced CNV model and human wet AMD patients. It has been also observed that endothelial precursor cells (EPCs) are partially recruited by the chemokine stromal-derived factor-1 (SDF-1) and its receptor CXCR4 at the site of neovascularization³⁶, which further stimulates neovascularization in the CNV. CXCR4 is expressed on EPCs and mature endothelial cells^{36d}. Oxidative stress has also had an impact on the recruitment of these EPCs at the site of CNV lesions. Moreover, it can promote the differentiation of EPCs to endothelial cells at the site of CNV upon activation^{36d}. Therefore, we want to evaluate the therapeutic effect of GCCNPs on the inhibition of these oxidative stress related pro-angiogenic responses. We found increased levels of 4-FINE, VEGF and CXCR4 expressions in the RPE/choroidal tissues compared to that of wild type (without laser) controls (FIG. 8C). Whereas the intravitreal injection of GCCNPs were able to attenuate the expression of these protein levels compared to the untreated controls (laser treated only), they remain comparable to the wild type (without laser) as demonstrated in FIG. 8C. The quantitative assessments (FIG. 8D) revealed that these laser-induced CNV associated 4-FINE, VEGF, and CXCR4 were significantly reduced in GCCNP injected RPE/choroidal samples compared to the untreated controls at 14 days post-laser treatments.

### GCCNPs Tend to Accumulate at Laser-Induced CNV Lesion Sites and Prefer to Stay in RPE in WT Mice Eyes

We also examined CNV targeting efficiency of GCCNPs by labeling these NPs with Alexa Fluor® 488 5-sulfodichlorophenol ester (A30052, Molecular probes) using standard bioconjugation technique. We injected 2 μl of GCCNPs-488 (conjugate, 0.4 μg/μl) to the intravitreal space of laser treated eyes (following same protocol as GCCNP injections) and these eyes were collected at 72 hrs after injection to prepare choroidal flat mounts. The flat mounts were screened and we found that laser-CNV lesions showed green fluorescence of GCCNPs-488 (FIG. 8E) that clearly revealed CNV targeting activity of the novel GCCNP formulation. In addition, in WT mice eyes (without laser damage), our data showed that GCCNPs prefer to stay in RPE cells after intravitreal injection.

### GCCNPs Don't Promote Toxicity to Ocular Tissues

Finally, we were concerned whether GCCNPs elicit any toxicity to the ocular tissues. To address this issue, we have injected 2 μl of GCCNPs (0.4 μg/μl) inside the vitreous space. These eyes were not treated with laser. After 72 hrs of injection, the eyes were enucleated, sectioned, and processed for H&E staining and immunohistochemistry to study the effect of GCCNPs on the morphometric analysis and microphage/microglia protein marker F4/80 expression into the retina. The morphometric analysis revealed that GCCNP delivery has no effect on the retinal degeneration in either saline or GCCNP injected retina as confirmed by the measurements of ONL thickness in superior and inferior hemispheres. Moreover, there was no such F4/80 immunoreactivity observed in saline or GCCNP injected retinas. In contrast, strong F4/80 expressions were detected in the lipopolysaccharide (LPS, Escherichia coli, Sigma) injected (at sub-retinal space) positive control that stimulates immune responses.

### Discussion

In this study, a nanoceria formulation was developed. This system exhibited excellent water solubility and robust antioxidant activities with potential real time tracing of the therapeutic responses. Our results showed that delivery of GCCNP suppressed H₂O₂-induced EC migration and tube formation in vitro, inhibited pro-angiogenic VEGF, oxidative marker protein (4-HNE adduct), chemokine CXCR4 receptor expressions, and subsequently attenuated CNV in a murine model of wet AMD. Therefore, this system could be especially beneficial for AMD patients for whom standard options won't work or are not available. Beyond its potential use in AMD, the formulation will also establish a widely applicable method for oxidative stress-related diseases, such as neurological disorders, aging, and cancer.

AMD is a multifactorial, progressive and complex disorder where age plays a key role. With age over 65 years, oxidative stress in the retina/RPE/choroid increases, antioxidant activity decreases, and therefore, pathological level of ROS increases³⁷, which further generates different pathological conditions by cumulative oxidations of proteins, lipids, and DNAs^{19b, 21, 24, 26-27, 38}. ROS induced oxidation generates ω-(2-carboxyethyl) pyrrole (CEP) protein adducts, derived from docosahexaenoate (DHA)-containing lipids, were found to be more abundant in samples of AMD human donors compared to that of the normal human being³⁹. Several mouse models established that oxidative stress plays a significant role in the pathogenesis of AMD^{25, 27, 40}. Under physiological conditions, ROS can act as a survival factor whereas due to over expression or poor endogenous defense system, ROS may lead to oxidative stress and damage to the retina/RPE/Br membrane, which induces death signaling pathways to generate severe pathological conditions like AMD. It is well established in literature that oxidative stress promotes angiogenesis by stimulating VEGF⁴¹. Therefore, pathological levels of ROS can stimulate CNV by promoting a pro-angiogenic environment. Furthermore, previous studies demonstrated the protective role of antioxidants on retinal tissues by inhibiting neovascularization^{40a, 41}. VEGF is a significant pro-angiogenic factor tissues by inhibiting neovascularization that stimulates CNV⁴². Earlier studies also demonstrated that stimulated VEGF can further activate NADPH oxidase to increase the ROS that can promote migration and proliferation of ECs⁴³. To this end, our hypothesis was to inhibit angiogenesis by scavenging free radicals in a laser-CNV murine model, a classical model of wet AMD, as this strategy may reduce the burden of cumulative oxidative damages and their consequent pro-angiogenic responses.

CNPs have been established to be a promising candidate for healing the oxidative stress associated disorder. However, most of the current efforts are concerned with the water dispersible bared CNPs for their potential in vivo therapeutic efficacies^{4-7, 40a}. Therefore, we became more interested in the development of water soluble, stable as well as biocompatible CNPs with hydrophilic shell. Pure inorganic nanoparticles loaded with GC, a chitosan derivative, have been extensively studied to improve their therapeutic efficiencies⁴⁴. Intravitreal injections of positively charged GC nanoparticles have demonstrated invasion through the vitreous and significant distributions in the retina⁴⁵. Moreover, GC demonstrated natural free radical scavenging activity due to the inherent amino groups as found in an earlier study⁴⁶. Hence, we became interested in combining the auto-regenerative bared CNPs with natural GC antioxidant polymer into the same platform to achieve a water soluble, stable and biocompatible nano-formulation, which improved antioxidant property of bared CNPs itself as evidenced by ORAC and EPR studies. In the current study, the hydroxyl groups of the GC polymer may be chemically bonded to the cerium oxide surface during coating as mentioned in the earlier study with the dextran and glucose stabilized CNPs¹². Herein, the integral glycol groups are acting as an anti-fouling system, which is also responsible for the aqueous solubility of GCCNPs without any additives (e.g. acid that is needed to solubilize chitosan polymer in water). Surface of the CNPs were successfully coated with GC as confirmed by the TGA. The engagement of hydroxyl groups with ceria leaves amine groups available to take part in further potential conjugations as we have seen in the GCCNPs-488 labeling study. These amine groups were also responsible for the generation of slightly positive surface charge and available to achieve free radical scavenging activity of GCCNPs.

The cumulative oxidative stress and damage of RPE as well as its barrier breakdown are directly correlated to invasions of choroidal vessels and the pathogenesis of AMD^{33b, 33c}. We had observed that these NPs were safe towards the human RPE cells as well as to the retina. GCCNPs scavenged intracellular free radicals as induced by H₂O₂ in the ARPE19 cells. We didn't observe any change in morphology of human RPE cells upon treatment with the different doses of GCCNPs (data not shown). Furthermore, GCCNPs were not developing any ROS in the ARPE19 cells under the experimental condition. In AMD patients and animal models of CNV, VEGF is overexpressed in the RPE⁴⁷. This VEGF is associated with rupture of barrier junctions, cytotoxicity of RPE tissues, and promotion of CNV^{33b, 33d}. In our experiments, H₂O₂₋induced oxidative stress in human RPE cells could generate VEGF, which was further down regulated by GCCNPs via scavenging intra cellular ROS. This VEGF inhibition is concomitant with scavenging ROS and oxidative stress reduction, which may prevent VEGF mediated injuries in CNV.

Angiogenesis is important for the growth of CNV and VEGF is the primary angiogenic inducer. VEGF mediated downstream signaling promotes cell migration, tube formation and finally angiogenesis. Interestingly, GCCNPs repressed EC migration and tube formation in HUVEC cells, consistent with earlier observation with bared CNPs^{11, 17} and demonstrating its anti-angiogenic activity in vitro¹¹. The GCCNPs also inhibited the VEGF induced by H₂O₂ in a dose dependent manner in HUVEC cells. Therefore, GCCNPs might reduce the pathological ROS that induces VEGF in the HUVEC cells and further prevent the pathological VEGF mediated injuries in CNV. To conclude and confirm the anti-angiogenesis effect of GCCNPs, we moved from the culture models that were adopted for their simplicity to a laser-induced CNV mice model (well adopted classical model) to mimic the complex tissue environment as observed in wet AMD. The current study demonstrates that GCCNP administrations attenuate CNV via the inhibitory effect on oxidative stress (4-HNE), and VEGF. We further observed that GCCNPs injections also suppressed CXCR4 expression. ROS has been demonstrated to be an important factor in inducing CXCR4 expression⁴⁸. Earlier studies also demonstrated that VEGF stimulates CXCR4 expressions and progression of angiogenesis ^{36c, 49}. CXCR4 is one of the important factors in the recruitment of EPCs at CNV, which contributes to the pathogenesis of CNV^{36a}. CXCR4 signaling also stimulates the VEGF induced angiogenesis in the retina^{36a}. CXCR4 antagonist therapy causes blockage to the CXCR4 that results in suppression of retinal neovascularization^{36a, 36c} and angiogenesis. Therefore, inhibition of CXCR4 explored as an important therapeutic modality to reduce the growth of neovasculatures. Interestingly, we detected significantly decreased VEGF and CXCR4 expression in the GCCNP-treated mice eyes. Moreover, the single intravitreal injection was enough to reduce the CNV damaged areas. The eye is prone to oxidative stress due to assembly of huge amounts of PUFA and high rates of oxygen metabolism. GCCNP administration could reduce the lipid oxidations by inhibition of ROS production. ROS-induced cumulative oxidative damages, which contribute to the pathogenesis of AMD, were reduced by a significant amount as observed in the western blot of laser-induced RPE/choroid CNV tissues. Retinal tissues were also safe towards the GCCNPs in terms of morphology of retina and microphage/microglia recruitments. Thus, GCCNPs explored attenuation of laser-induced CNV without any associated ocular toxicity. To the best of our knowledge, this is the first report of the exploitation of water-soluble GCCNPs for successful treatment of wet AMD both in vitro and in vivo models. Altogether, these comprehensive studies established that GCCNPs were able to target and heal laser-induced CNV lesions without having any significant toxicity to the retina.

The water-soluble nanoceria (GCCNP) has significantly enhanced the solubility of nanoceria in pure water, and thus has significantly reduced its potential toxicity. These GC coated ceria nanoparticles may be potent candidate for therapeutic use and can also be presented for FDA approval. In the present invention, the catalytic and auto-regenerative properties of ceria nanoparticles remained unaffected by the use of GC coating. Instead, the free radical scavenging property of GC reflected in an additive property to that of ceria NPs. These GCCNPs are easy to handle in lyophilized condition to anywhere at ambient condition for biomedical applications. Our study revealed that a single intravitreal injection of a novel and biocompatible auto-regenerative antioxidant GCCNP can alter the oxidative stress associated lesions in a laser-induced CNV mice model. Due to the presence of amine groups on the surface, these nanoparticles can be easily modified with different targeting agents, protein, aptamers, oligonucleotides, peptides, antibodies, an/or small organic molecules.

### Materials and Methods

### Animals

We used adult 6-8 weeks old C57BL/6J male mice (Jackson Laboratory, Me.) for laser-induced CNV studies. All experiments were carried out and animals were maintained in accordance with the Association for Research in Vision and Ophthalmology (ARVO) statements for the use of animals in ophthalmic and vision research, and the guidelines of the University of North Carolina at Chapel Hill animal care and use committee.

### Synthesis and Characterizations of Engineered Water Soluble GCCNPs

The bared or uncoated CNPs (BCNPs) and coated GCCNPs were prepared by NH₄OH precipitation method. To prepare GCCNPs, a 0.2 ml solution of 0.7 (M) CeCl₃, 7H₂O (Sigma) was added to the 5 ml of 2% (w/v) of glycol chitosan (GC, Sigma, degree of polymerization ≥400) solution under constant stirring at room temperature for 20 min. 0.1 ml of concentrated ammonium hydroxide (Sigma, 28-30%) was slowly added to the mixture. Thereafter, solution was centrifuged at 5000 rpm for 10 min at room temperature to avoid any aggregation and the clear supernatant was collected. This supernatant was then dialyzed using slide-A-lyzer dialysis cassettes (Thermo Fisher Scientific, 20 kDa MWCO cut off) for 2 days at room temperature against nanopure water. After dialysis, the solution was neutralized using acetic acid (Sigma, >99%) to physiological pH 7.4. BCNPs were prepared parallel to GCCNPs following the same methodology. The BCNPs and GCCNPs solutions were then lyophilized and stored at −20° C. for further use. For further use, the lyophilized material was taken in water (endotoxin free, HyClone™ Water, GE healthcare) and warmed at 75° C. for 5 mins to get the clear and transparent solution of aqueous soluble GCCNPs. These water soluble GCCNPs and water insoluble BCNPs were further ultra-sonicated before each in vitro and in vivo use. The GC polymer conjugates with the CNPs via hydroxyl groups as depicted earlier with the dextran coating studies in polyhydroxyl solution¹². GC prevents CNPs from crystallization or further aggregation even after the use of lyophilized GCCNPs in ultrapure and sterile water.

The nanoparticles were characterized by different physicochemical techniques. The morphology, size, shape and energy-dispersive X-ray spectroscopy (EDS) of synthesized NPs were determined by transmission electron microscopy (TEM) on JEOL 2010E-FasTEM at the Chapel Hill Analytical and Nanofabrication Laboratory (CHANL, UNC-CH); the hydrodynamic diameters and surface charge of the NPs were measured by dynamic light scattering (DLS) and zeta potential respectively on a Nano-ZS zeta sizer (Malvern, USA); Ce⁺³/Ce⁺⁴ patterns were determined by X-ray photoelectron spectroscope (XPS, Kratos Axis Ultra DLD, CHANL-UNC); the concentrations of NPs were calculated by inductively coupled plasma atomic emission spectroscopy (ICP, Varian 820-MS-ICP-MS, CHANL-UNC); the Raman spectrum were collected on a CRAIC micro-spectrophotometer (CHANL-UNC); the thermogravimetric analyses (TGA) were carried out on TGA Q50 (TA instrument, USA) at the nanomedicines characterization core facility at the University of North Carolina at Chapel Hill (UNC-CH).

### Determination of Oxygen Radical Absorbance Capacity (ORAC)

The ORAC of the GCCNPs and BCNPs were determined by previously described protocol⁵⁰.

### Materials and Sample Preparation for Electron Paramagnetic Resonance (EPR) Experiments

DMPO (5,5-dimethyl-1-pyrroline N-oxide) was purchased from SigmaAldrich (Milwaukee, Wis.). Other chemicals were purchased from VWR International (West Chester, Pa.). Stock solutions were prepared from deionized water (Milli-Q, Millipore Synergy® UV Water Purification System, Merck Millipore, Billerica, Mass.), 1 M DMPO, 1 mM Fe(II) sulfate, and 10 mM hydrogen peroxide. Stock solutions of NPs were prepared in at 40 μM concentration. For spin-trapping experiments the samples were prepared in the following order: 2.5 μl of stock DMPO solution was added to 12.5 μl of iron sulfate, followed by addition of 25 μl of the samples under the study (or water in the control experiment) and mixed. Next, the reaction was initiated by addition of 12.5 μl of stock solution of hydrogen peroxide and the timer was started at that moment. In experiments designed to check stability of the spin-adduct in the presence of nanoparticles, mixing was done in the following order: 2.5 μl of stock DMPO solution was added to 12.5 μl of iron sulfate and mixed. In the final step the reaction was initiated by addition of 12.5 μl of hydrogen peroxide and the timer was started at that moment. After incubation of the mixture for 5 min to form hydroxyl radical adduct, 25 μl of a sample under the study (or water in the control experiments) was added, mixed, and liquid was drawn into a capillary for EPR experiments. The ratio of DMPO:FeSO₄:H₂O₂ in the final mixtures was selected based on conditions known to produce the most stable spin-adduct EPR signal.

### EPR Spin-Trapping Experiments

Liquid samples were drawn into a glass capillary (0.81 mm i.d.×1.42 mm o.d., Jaguar Industries, Inc., Haverstraw, N.Y.) to form a column of liquid of about 6 cm long, the capillary was sealed by Critoseal™ capillary tube sealant (Leica Microsystems, Inc., Buffalo Grove, Ill.), and inserted into a standard 3 mm i.d.×4 mm o.d. quartz EPR tube (Wilmad-LabGlass, Vineland, N.J.). Room temperature CW EPR measurements were conducted using Bruker ELEXSYS E500 spectrometer (Bruker Biospin, Billerica, Mass.) operating at approximately 9.867 GHz (X-band). The data acquisition parameters were set as follows: modulation amplitude, 1 G; microwave power, 2 mW; scan width, 100 G; sweep time, 41.95 s; time constant, 40.96 ms, conversion time 40.97 ms. Typically, a sequence of 20 spectra was collected with 230 s interval between the spectra. The peak-to-peak amplitude of the line corresponding to the second from the low field nitrogen hyperfine transition was measured. This amplitude was assumed to be proportional to the concentration of the spin adduct because according to least-squares simulations (not shown) the EPR line shape did not change in the course of experiments. All mixing and incubations of the sample with spin trap solutions as well as EPR spectra averaging were carried out at room temperature (294 K).

### Cell Viability Assay

The ARPE19 cells were grown with 100% confluency. The cells were trypsinised (0.05% Trypsin-EDTA, GIBCO) and viable cells were counted using a hemocytometer. 1×10⁵ cells were seeded in each well of a 96-well microtiter plate (Corning, USA) and incubated overnight in a humidified atmosphere (e.g. 37° C., 5% CO₂). The next day, the medium was replaced with the fresh media and the cells were treated with BCCNPs and GCCNPs with varying concentrations to keep the untreated control. The cells were maintained at 37° C. and 5% CO₂ for 24 hrs. After the incubation period, the media was discarded and wells were washed with 1× Dulbecco's phosphate-buffered saline (DPBS) before adding fresh culture medium (100 μl per well). Following the manufacturer protocol, 10 μl of WST-1 (Life Science Research, USA), a cell proliferation reagent, was added to each well of the cells (1:10 final dilution). The cells were incubated for 2 hrs at humidified atmosphere (e.g. 37° C., 5% CO₂). Then the 96-well plate was shaken for 1 min on a shaker and the cell viability was determined using a microplate reader (Molecular Devices Spectramax M5) at 450 nm.

### ROS Measurement

Intracellular ROS was measured by oxidation of 2,7-dichlorodihydrofluorescein diacetate (DCFH-DA; Sigma) to highly fluorescent 2′,7′-dichlorofluorescein (DCF) intracellularly. This is a sensitive marker of cellular oxidation processes and determines the response of ROS rapidly in cells. DCFH-DA can penetrate though the cell membrane and readily deacetylate to DCFH which is further oxidized by intracellular ROS to highly fluorescent DCF inside the cells. 1×10⁴ ARPE19 cells were seeded per well on a 96-well plate and allowed to grow overnight in Dulbecco's modified Eagle's medium (DMEM), F12 (Invitrogen) with 10% fetal bovine serum (FBS). The next day, the cells were washed with 1× DPBS (3 times; 10 min) and treated with 0.5 mM H₂O₂ in serum free DMEM/F12 for 1 hr. The cells were then washed with 1× DPBS (3 times; 10 min) and incubated with DCFH-DA (50 μM) for 45 min at 37° C. Finally, the fluorescence intensity of DCF was determined with maximum excitation (at 485 nm) and emission spectra (at 530 nm).

### Tube Formation Assay

The in vitro tube formation assay was determined as performed earlier⁵¹. In brief, 60 μl of corning Matrigel matrix (Fisher scientific, USA) was placed per well of 96 well plate and incubated at 37° C. for 45 mM to allow basement membrane to form gel. 100 μl of 5×10⁴ HUVEC cells were transferred to each well with or without treatments with varying concentrations of GCCNPs. 2-methoxyestradiol (2-Me) was taken as negative control (10 nM). The plate was incubated for 8 hrs at 37° C. with 5% CO₂ and tube like structures were imaged and analyzed at 50× magnification in bright field Axio Observer.D1 inverted microscope (Carl Zeiss, Norway). Each experiment was carried out in triplicate.

### Migration Assay

In vitro HUVEC cell migration assay or scratch assay was performed as mentioned earlier^{51a, 52}. In brief, 5×10⁵ HUVEC cells were seeded in each well of 24 well plates and incubated overnight. The next day, 2 separate straight line scratches (per well) were made on the monolayer of HUVEC cells with sterile 200 μl pipette tip and the debris were cleaned by gentle washing with 1× DPBS. Different concentrations of GCCNPs were added to the 500 of media. 10 nM of 2-methoxyestradiol (2-Me) were taken as negative controls in this study. The plate was then incubated in tissue culture incubator at 37° C. for 24 hrs. Images of the scratches were acquired at 50× magnification before and after incubations (0 hr and 24 hrs) and further analyzed using Axio Observer.D1 inverted microscope (Carl Zeiss, Norway). Each experiment was made in triplicate.

### Fundus or Fluorescein Angiography

Two weeks after laser treatments, fluorescein angiography (FA) was carried out following the earlier protocol^{51a, 53}. In brief, the mice were fully anesthetized and the eyes were dilated and then a drop of lubricant gel was placed on each eye. These mice were then placed on the platform of the Micron III fundoscopy system (Phoenix Research laboratories, Pleasanton, Calif.). On getting a clear bright field image, 1% AK-FLUOR (Alcon, 100 cc/20 g mice) was intraperitoneally injected. The images were processed using StreamPix software.

### Laser-Induced CNV

Laser-induced CNV lesions were carried out following the standard protocol³⁵. In brief, animals were first anesthetized by intramuscular injection of a mixture of ketamine (85 mg/kg) and xylazine (14 mg/kg) (Butler Schein Animal Health, Dublin, Ohio), and the pupils were dilated by a drop of 1% tropicamide (Bausch & Lomb Inc., Tampa) and allowed 2-5 mins for complete dilation. A drop of Genteal lubricant gel (Alcon) was placed on the eye to avoid any eye dehydration. 3 laser photocoagulations (532 nm, 440 mW, 80 ms) were implemented to each eye surrounding the optic nerve and focusing on the Bruch's membrane (Br) which was confirmed by the air bubble sign of Br rapture using a Micron III Retinal Image-Guided Laser System (Phoenix Research laboratories, Pleasanton, Calif.) at 0300, 0900 and 1200 hours to generate choroidal neovascularization (CNV).

On the same day of photocoagulation, intravitreal injections were carried out following earlier protocols⁵⁰⁻⁵¹. In brief, the sclera of each eye was carefully punctured using a 30-gauge needle to make a clear hole. A 35-gauge needle attached to a 10 μl Nanofil syringe (World Precision Instruments, Sarasota, Fla., USA) was then gently inserted through the puncture hole at a 45° angle with respect to the scleral surface and the visualization aided by use of an operating microscope (Carl Zeiss Surgical, Incorporated, Thornwood, N.Y., USA) to slowly deliver 2 μl of GCCNPs (0.4 ng/μl) and/or saline solutions into the vitreous cavity. Triple antibiotic (Equate, Wal-Mart, Bentonville, Ark., USA) ointment was dropped on the eye surface right after the surgery to avoid any post-surgical infection. Mouse was laid on a 37° C. warm bed until they were fully awake. The mice were kept in a temperature controlled room with cyclic light (12 L:12 D) conditions for another 14 days as an end point of our study.

### Statistics

The current results were presented as the means of ±SEM of at least 3 independent experiments. The figures and data analysis were carried out with GraphPad Prism 5.0 software (La Jolla, Calif., USA). The figures were finally organized in Photoshop CS5 software. The results were analyzed by Student t-test between two independent groups. Multiple group comparisons were analyzed by one-way and 2-way ANOVA as appropriate. The calculations with P<0.05 were considered statistically significant.

### Cell Culture

Human retinal pigment epithelial (ARPE-19) and human umbilical vein ECs (HUVEC) cell lines were purchased from tissue culture facility (TCF) of the University of North Carolina at Chapel Hill. The cell cultures were maintained in Ham's F-12 nutrient medium (DMEM/F-12, GlutaMAX™ supplement, Gibco; added with 10% fetal bovine serum and 1M HEPES for ARPE19 cell line) and HuMEC (added with 5% FBS; for HUVEC cell line) supplemented with Antibiotic-Antimycotic (1×, Gibco, Life technologies).

### Western Blot Analysis

Western blot was carried out following the standard protocol as mentioned previously. Cell homogenates from ARPE19/HUVEC or isolated RPE/choroid complexes were obtained in RIPA buffer (Pierce, Thermo Scientific) with 1× EDTA-free protease inhibitor cocktail (Roche) on ice. Extracted proteins were quantified using BCA protein assay (Biorad). 30 μg of protein extract was mixed with sample buffer and fresh 2-marcaptoethanol and SDS, followed by heating at 95° C. for 5 min. These equal amounts of protein samples were individually loaded on 10% SDS-polyacrylamide gel and after completing the electrophoresis, proteins in the gel were transferred to the PVDF membrane. The membrane was blocked with 5% milk in 1× PBST and incubated with rabbit anti-VEGF-A (ab46154, abcam; 1:1000) or rabbit polyclonal anti-4-HNE (ab46545, abeam, 1:1500) or mouse monoclonal anti-CXCR4 (60042-1-Ig, Proteintech. IL) in 5% milk in 1× PBST buffer overnight at 4° C. The next day, the membrane was washed with 1× PBST (3 times, 10 min each) and then incubated with goat anti-rabbit and anti-mouse IgG antibodies (sc-2030 and sc-2031 respectively Santa Cruz) at 1:20,000 for 2 hrs at room temperature followed by washing with 1× PBST (3 times, 10 min each). The washed membrane was developed and visualized by Super Signal West Dura Extended Duration Substrate (Thermo Scientific, Rockford, Ill.) using manufacturer's protocol. Blots were analyzed by ChemiDoc™ MP imaging system (Bio-rad) and densitometric analyses were determined using Image Lab software v4.1 (Bio-rad). The pixel densities in each band were normalized to the corresponding amount of mouse housekeeping beta-actin (A3854, Sigma, 1:50,000).

### Determination of Outer Nuclear Layer (ONL) Thickness

ONL thickness was determined by the protocol as demonstrated in a previous report (36). In brief, at 14 days of laser treatments, mice were euthanized and superior position was marked, eyes were enucleated and fixed in 10% formalin (formalde-fresh, cat no. SF93-4, Fisher). The paraffin sections were stained with hematoxylin and eosin (H&E) and images were analyzed by Axio Observer.D1 microscope (Carl Zeiss, Norway) at 200× magnification. Images for demonstrating ONL thickness were collected from 3 separate eyes per group, beginning at the optic nerve head (ONH) and proceeding towards periphery of superior and inferior hemispheres at 435 mm intervals.

### Immunohistochemistry

Immunohistochemistry was performed as demonstrated in an earlier protocol (40). In brief, enucleated eyes were punctured to make a clear hole and were fixed in 4% PFA for 1 hr at room temperature. The cornea and lens were removed from the dissected eye and placed in 4% PFA for 2 hrs at room temperature and then treated with sucrose gradient (10%-25%) in 1× PBS, embedded in optimal cutting temperature (OCT) compound (Tissue plus, Fisher) and frozen immediately in dry-ice; the posterior eye sections were cut with 10-mm-thickness on slides. Slides were blocked in donkey blocker, and stained with F4/80 (M-300) primary antibody (sc-25830, Santa Cruz) overnight at 4° C. The slides were then washed with 1 ×PBST (3 times; 10 min each), incubated with Alexa Fluor® 555 donkey anti-rabbit IgG (H+L) secondary antibody (1:1000, A-31572, Invitrogen) for 2 h at room temperature, followed by washing as earlier and then mounted with DAPI-containing mounting media (VECTASHIELD; Vector Laboratories, Calif.) for further imaging and acquisitions using Zeiss LSM 710 spectral confocal laser scanning microscope.

### Quantification of CNV

14 days after the laser treatments, mice were taken for fundus and OCT analyses before sacrifice. Choroidal flat mounts were consequently prepared following standard protocol (40). The eyes were fixed in 4% paraformaldehyde for 2 hrs at room temperature. The eyes were then dissected, muscles, cornea. and lens were carefully removed, and finally eye cups were taken in 1× PBS overnight at room temperature. The next day, the retina/RPE/choroid/sclera complexes were carefully flattened by making 4-5 radial incisions for each eye cup. Retina was gently removed from the underlying RPE/choroid/sclera in order to make choroidal flat-mount on glass slide. The flat-mount was then blocked with normal donkey blocker for 1 hr at room temperature and incubated with Alexa Fluor®488 conjugated isolectin GS-IB4 (Invitrogen, Cat. No. 121411) at a dilution of 1:200 overnight at 4° C. Each flat-mount was finally washed with 1× PBS and mounted with antifade mounting media (VECTASHIELD; Vector Laboratories, Calif.) to preserve the fluorescence. Using Zeiss LSM 710 spectral confocal laser scanning microscope, the choroidal flat-mounts were analyzed. The CNV development was quantified by considering positive stained area of isolectin as areas of CNV area at each laser lesion and NIH ImageJ software was used as a measuring tool.

### REFERENCES

1. Esch, F.; Fabris, S.; Zhou, L.; Montini, T.; Africh, C.; Fornasiero, P.; Comelli, G.; Rosei, R., Electron localization determines defect formation on ceria substrates. Science 2005, 309 (5735), 752-5.
2. (a) Li, Y.; He, X.; Yin, J. J.; Ma, Y.; Zhang, P.; Li, J.; Ding, Y.; Zhang, J.; Zhao, Y.; Chai, Z.; Zhang, Z., Acquired superoxide-scavenging ability of ceria nanoparticles. Angew Chem Int Ed Engl 2015, 54 (6), 1832-5; (b) Heckert, E. G.; Karakoti, A. S.; Seal, S.; Self, W. T., The role of cerium redox state in the SOD mimetic activity of nanoceria. Biomaterials 2008, 29 (18), 2705-2709.
3. Pirmohamed, T.; Dowding, J. M.; Singh, S.; Wasserman, B.; Heckert, E.; Karakoti, A. S.; King, J. E.; Seal, S.; Self, W. T., Nanoceria exhibit redox state-dependent catalase mimetic activity. Chem Commun (Camb) 2010, 46 (16), 2736-8.
4. (a) Chen, J. P.; Patil, S.; Seal, S.; McGinnis, J. F., Rare earth nanoparticles prevent retinal degeneration induced by intracellular peroxides. Nature Nanotechnology 2006, 1 (2), 142-150; (b) Kong, L.; Cai, X.; Zhou, X.; Wong, L. L.; Karakoti, A. S.; Seal, S.; McGinnis, J. F., Nanoceria extend photoreceptor cell lifespan in tubby mice by modulation of apoptosis/survival signaling pathways. Neurobiol Dis 2011, 42 (3), 514-23.
5. Zhou, X.; Wong, L. L.; Karakoti, A. S.; Seal, S.; McGinnis, J. F., Nanoceria inhibit the development and promote the regression of pathologic retinal neovascularization in the Vldlr knockout mouse. Plos One 2011, 6 (2), e16733.
6. Kyosseva, S. V.; Chen, L.; Seal, S.; McGinnis, J. F., Nanoceria inhibit expression of genes associated with inflammation and angiogenesis in the retina of Vldlr null mice. Experimental eye research 2013, 116, 63-74.
7. Cai, X.; Sezate, S. A.; Seal, S.; McGinnis, J. F., Sustained protection against photoreceptor degeneration in tubby mice by intravitreal injection of nanoceria. Biomaterials 2012, 33 (34), 8771-81.
8. Das, M.; Patil, S.; Bhargava, N.; Kang, J. F.; Riedel, L. M.; Seal, S.; Hickman, J. J., Auto-catalytic ceria nanoparticles offer neuroprotection to adult rat spinal cord neurons. Biomaterials 2007, 28 (10), 1918-25.
9. Dowding, J. M.; Song, W.; Bossy, K.; Karakoti, A.; Kumar, A.; Kim, A.; Bossy, B.; Seal, S.; Ellisman, M. H.; Perkins, G.; Self, W. T.; Bossy-Wetzel, E., Cerium oxide nanoparticles protect against Abeta-induced mitochondrial fragmentation and neuronal cell death. Cell Death Differ 2014, 21 (10), 1622-32.
10. Kim, C. K.; Kim, T.; Choi, I. Y.; Soh, M.; Kim, D.; Kim, Y. J.; Jang, H.; Yang, H. S.; Kim, J. Y.; Park, H. K.; Park, S. P.; Park, S.; Yu, T.; Yoon, B. W.; Lee, S. H.; Hyeon, T., Ceria nanoparticles that can protect against ischemic stroke. Angew Chem Int Ed Engl 2012, 51 (44), 11039-43.
11. Giri, S.; Karakoti, A.; Graham, R. P.; Maguire, J. L.; Reilly, C. M.; Seal, S.; Rattan, R.; Shridhar, V., Nanoceria: a rare-earth nanoparticle as a novel anti-angiogenic therapeutic agent in ovarian cancer. Plos One 2013, 8 (1), e54578.
12. Karakoti, A. S.; Kuchibhatla, S. V. N. T.; Babu, K. S.; Seal, S., Direct synthesis of nanoceria in aqueous polyhydroxyl solutions. Journal of Physical Chemistry C 2007, 111 (46), 17232-17240.
13. Perez, J. M.; Asati, A.; Nath, S.; Kaittanis, C., Synthesis of biocompatible dextran-coated nanoceria with pH-dependent antioxidant properties. Small 2008, 4 (5), 552-556.
14. Zhai, Y. W.; Zhou, K. B.; Xue, Y.; Qin, F.; Yang, L. M.; Yao, X., Synthesis of water-soluble chitosan-coated nanoceria with excellent antioxidant properties. Rsc Adv 2013, 3 (19), 6833-6838.
15. Asati, A.; Santra, S.; Kaittanis, C.; Nath, S.; Perez, J. M., Oxidase-like activity of polymer-coated cerium oxide nanoparticles. Angew Chem Int Ed Engl 2009, 48 (13), 2308-12.
16. Li, M.; Shi, P.; Xu, C.; Ren, J. S.; Qu, X. G., Cerium oxide caged metal chelator: anti-aggregation and anti-oxidation integrated H2O2-responsive controlled drug release for potential Alzheimer's disease treatment. Chem Sci 2013, 4 (6), 2536-2542.
17. Lord, M. S.; Tsoi, B.; Gunawan, C.; Teoh, W. Y.; Amal, R.; Whitelock, J. M., Anti-angiogenic activity of heparin functionalised cerium oxide nanoparticles. Biomaterials 2013, 34 (34), 8808-18.
18. Kwon, H. J.; Cha, M. Y.; Kim, D.; Kim, D. K.; Soh, M.; Shin, K.; Hyeon, T.; Mook-Jung, I., Mitochondria-Targeting Ceria Nanoparticles as Antioxidants for Alzheimer's Disease. ACS Nano 2016, 10 (2), 2860-70.
19. (a) Jager, R. D.; Mieler, W. F.; Miller, J. W., Age-related macular degeneration. The New England journal of medicine 2008, 358 (24), 2606-17; (b) Holz, F. G.; Schmitz-Valckenberg, S.; Fleckenstein, M., Recent developments in the treatment of age-related macular degeneration. The Journal of clinical investigation 2014, 124 (4), 1430-8; (c) Bird, A. C., Therapeutic targets in age-related macular disease. Journal of Clinical Investigation 2010, 120 (9), 3033-3041.
20. Fritsche, L. G.; Igl, W.; Bailey, J. N.; Grassmann, F.; Sengupta, S.; Bragg-Gresham, J. L.; Burdon, K. P.; Hebbring, S. J.; Wen, C.; Gorski, M.; Kim, I. K.; Cho, D.; Zack, D.; Souied, E.; Scholl, H. P.; Bala, E.; Lee, K. E.; Hunter, D. J.; Sardell, R. J.; Mitchell, P.; Merriam, J. E.; Cipriani, V.; Hoffman, J. D.; Schick, T.; Lechanteur, Y. T.; Guymer, R. H.; Johnson, M. P.; Jiang, Y.; Stanton, C. M.; Buitendijk, G. H.; Zhan, X.; Kwong, A. M.; Boleda, A.; Brooks, M.; Gieser, L.; Ratnapriya, R.; Branham, K. E.; Foerster, J. R.; Heckenlively, J. R.; Othman, M. I.; Vote, B. J.; Liang, H. H.; Souzeau, E.; McAllister, I. L.; Isaacs, T.; Hall, J.; Lake, S.; Mackey, D. A.; Constable, I. J.; Craig, J. E.; Kitchner, T. E.; Yang, Z.; Su, Z.; Luo, H.; Chen, D.; Ouyang, H.; Flagg, K.; Lin, D.; Mao, G.; Ferreyra, H.; Stark, K.; von Strachwitz, C. N.; Wolf, A.; Brandl, C.; Rudolph, G.; Olden, M.; Morrison, M. A.; Morgan, D. J.; Schu, M.; Aim, J.; Silvestri, G.; Tsironi, E. E.; Park, K. H.; Farrer, L. A.; Orlin, A.; Brucker, A.; Li, M.; Curcio, C. A.; Mohand-Said, S.; Sahel, J. A.; Audo, I.; Benchaboune, M.; Cree, A. J.; Rennie, C. A.; Goverdhan, S. V.; Grunin, M.; Hagbi-Levi, S.; Campochiaro, P.; Katsanis, N.; Holz, F. G.; Blond, F.; Blanche, H.; Deleuze, J. F.; Igo, R. P., Jr.; Truitt, B.; Peachey, N. S.; Meuer, S. M.; Myers, C. E.; Moore, E. L.; Klein, R.; Hauser, M. A.; Postel, E. A.; Courtenay, M. D.; Schwartz, S. G.; Kovach, J. L.; Scott, W. K.; Liew, G.; Tan, A. G.; Gopinath, B.; Merriam, J. C.; Smith, R. T.; Khan, J. C.; Shahid, H.; Moore, A. T.; McGrath, J. A.; Laux, R.; Brantley, M. A., Jr.; Agarwal, A.; Ersoy, L.; Caramoy, A.; Langmann, T.; Saksens, N. T.; de Jong, E. K.; Hoyng, C. B.; Cain, M. S.; Richardson, A. J.; Martin, T. M.; Blangero, J.; Weeks, D. E.; Dhillon, B.; van Duijn, C. M.; Doheny, K. F.; Romm, J.; Klaver, C. C.; Hayward, C.; Gorin, M. B.; Klein, M. L.; Baird, P. N.; den Hollander, A. I.; Fauser, S.; Yates, J. R.; Allikmets, R.; Wang, J. J.; Schaumberg, D. A.; Klein, B. E.; Hagstrom, S. A.; Chowers, I.; Lotery, A. J.; Leveillard, T.; Zhang, K.; Brilliant, M. H.; Hewitt, A. W.; Swaroop, A.; Chew, E. Y.; Pericak-Vance, M. A.; DeAngelis, M.; Stambolian, D.; Haines, J. L.; Iyengar, S. K.; Weber, B. H.; Abecasis, G. R.; Heid, I. M., A large genome-wide association study of age-related macular degeneration highlights contributions of rare and common variants. Nature genetics 2016, 48 (2), 134-43.
21. Gu, X.; Meer, S. G.; Miyagi, M.; Rayborn, M. E.; Hollyfield, J. G.; Crabb, J. W.; Salomon, R. G., Carboxyethylpyrrole protein adducts and autoantibodies, biomarkers for age-related macular degeneration. The Journal of biological chemistry 2003, 278 (43), 42027-35.
22. (a) Mitra, R. N.; Conley, S. M.; Naash, M. I., Therapeutic Approach of Nanotechnology for Oxidative Stress Induced Ocular Neurodegenerative Diseases. Adv Exp Med Biol 2016, 854, 463-9; (b) Li, Q.; Dinculescu, A.; Shan, Z.; Miller, R.; Pang, J.; Lewin, A. S.; Raizada, M. K.; Hauswirth, W. W., Downregulation of p22phox in retinal pigment epithelial cells inhibits choroidal neovascularization in mice. Molecular therapy: the journal of the American Society of Gene Therapy 2008, 16 (10), 1688-94.
23. (a) Ambati, J.; Atkinson, J. P.; Gelfand, B. D., Immunology of age-related macular degeneration. Nat Rev Immunol 2013, 13 (6), 438-51; (b) Roggia, M. F.; Imai, H.; Shiraya, T.; Noda, Y.; Ueta, T., Protective role of glutathione peroxidase 4 in laser-induced choroidal neovascularization in mice. Plos One 2014, 9 (6), e98864.
24. Beatty, S.; Koh, H.; Phil, M.; Henson, D.; Boulton, M., The role of oxidative stress in the pathogenesis of age-related macular degeneration. Surv Ophthalmol 2000, 45 (2), 115-34.
25. Hollyfield, J. G.; Bonilha, V. L.; Rayborn, M. E.; Yang, X.; Shadrach, K. G.; Lu, L.; Ufret, R. L.; Salomon, R. G.; Perez, V. L., Oxidative damage-induced inflammation initiates age-related macular degeneration. Nature medicine 2008, 14 (2), 194-8.
26. Ebrahem, Q.; Renganathan, K.; Sears, J.; Vasanji, A.; Gu, X.; Lu, L.; Salomon, R. G.; Crabb, J. W.; Anand-Apte, B., Carboxyethylpyrrole oxidative protein modifications stimulate neovascularization: Implications for age-related macular degeneration. Proc Natl Acad Sci USA 2006, 103 (36), 13480-4.
27. Du, H.; Sun, X.; Guma, M.; Luo, J.; Ouyang, H.; Zhang, X.; Zeng, J.; Quach, J.; Nguyen, D. H.; Shaw, P. X.; Karin, M.; Zhang, K., JNK inhibition reduces apoptosis and neovascularization in a murine model of age-related macular degeneration. Proc Natl Acad Sci USA 2013, 110 (6), 2377-82.
28. (a) Kuroki, M.; Voest, E. E.; Amano, S.; Beerepoot, L. V.; Takashima, S.; Tolentino, M.; Kim, R. Y.; Rohan, R. M.; Colby, K. A.; Yeo, K. T.; Adamis, A. P., Reactive oxygen intermediates increase vascular endothelial growth factor expression in vitro and in vivo. The Journal of clinical investigation 1996, 98 (7), 1667-75; (b) Chua, C. C.; Hamdy, R. C.; Chua, B. H., Upregulation of vascular endothelial growth factor by H2O2 in rat heart endothelial cells. Free radical biology & medicine 1998, 25 (8), 891-7.
29. Zamiri, R.; Ahangar, H. A.; Kaushal, A.; Zakaria, A.; Zamiri, G.; Tobaldi, D.; Ferreira, J. M., Dielectrical Properties of CeO2 Nanoparticles at Different Temperatures. Plos One 2015, 10 (4), e0122989.
30. Voinov, M. A.; Pagan, J. O. S.; Morrison, E.; Smirnova, T. I.; Smirnov, A. I., Surface-Mediated Production of Hydroxyl Radicals as a Mechanism of Iron Oxide Nanoparticle Biotoxicity. J Am Chem Soc 2011, 133 (1), 35-41.
31. Villamena, F. A.; Hadad, C. M.; Zweier, J. L., Kinetic Study and Theoretical Analysis of Hydroxyl Radical Trapping and Spin Adduct Decay of Alkoxycarbonyl and Dialkoxyphosphoryl Nitrones in Aqueous Media. The Journal of Physical Chemistry A 2003, 107 (22), 4407-4414.
32. Fontmorin, J. M.; Burgos Castillo, R. C.; Tang, W. Z.; Sillanpaa, M., Stability of 5,5-dimethyl-1-pyrroline-N-oxide as a spin-trap for quantification of hydroxyl radicals in processes based on Fenton reaction. Water Research 2016, 99, 24-32.
33. (a) Kaczara, P.; Sarna, T.; Burke, J. M., Dynamics of H2O2 availability to ARPE-19 cultures in models of oxidative stress. Free radical biology & medicine 2010, 48 (8), 1064-70; (b) Bailey, T. A.; Kanuga, N.; Romero, I. A.; Greenwood, J.; Luthert, P. J.; Cheetham, M. E., Oxidative stress affects the junctional integrity of retinal pigment epithelial cells. Invest Ophthalmol Vis Sci 2004, 45 (2), 675-84; (c) Thurman, J. M.; Renner, B.; Kunchithapautham, K.; Ferreira, V. P.; Pangburn, M. K.; Ablonczy, Z.; Tomlinson, S.; Holers, V. M.; Rohrer, B., Oxidative stress renders retinal pigment epithelial cells susceptible to complement-mediated injury. The Journal of biological chemistry 2009, 284 (25), 16939-47; (d) Byeon, S. H.; Lee, S. C.; Choi, S. H.; Lee, H. K.; Lee, J. H.; Chu, Y. K.; Kwon, O. W., Vascular Endothelial Growth Factor as an Autocrine Survival Factor for Retinal Pigment Epithelial Cells under Oxidative Stress via the VEGF-R2/PI3K/Akt. Invest Ophth Vis Sci 2010, 51 (2), 1190-1197.
34. Yasuda, M.; Ohzeki, Y.; Shimizu, S.; Naito, S.; Ohtsuru, A.; Yamamoto, T.; Kuroiwa, Y., Stimulation of in vitro angiogenesis by hydrogen peroxide and the relation with ETS-1 in endothelial cells. Life Sci 1999, 64 (4), 249-58.
35. Lambert, V.; Lecomte, J.; Hansen, S.; Blacher, S.; Gonzalez, M. L.; Struman, I.; Sounni, N. E.; Rozet, E.; de Tullio, P.; Foidart, J. M.; Rakic, J. M.; Noel, A., Laser-induced choroidal neovascularization model to study age-related macular degeneration in mice. Nature protocols 2013, 8 (11), 2197-211.
36. (a) Lee, E.; Rewolinski, D., Evaluation of CXCR4 inhibition in the prevention and intervention model of laser-induced choroidal neovascularization. Invest Ophthalmol Vis Sci 2010, 51 (7), 3666-72; (b) Dong, A.; Shen, J.; Zeng, M.; Campochiaro, P. A., Vascular cell-adhesion molecule-1 plays a central role in the proangiogenic effects of oxidative stress. Proc Natl Acad Sci USA 2011, 108 (35), 14614-9; (c) Sengupta, N.; Afzal, A.; Caballero, S.; Chang, K. H.; Shaw, L. C.; Pang, J. J.; Bond, V. C.; Bhutto, I.; Baba, T.; Lutty, G. A.; Grant, M. B., Paracrine modulation of CXCR4 by IGF-1 and VEGF: implications for choroidal neovascularization. Invest Ophthalmol Vis Sci 2010, 51 (5), 2697-704; (d) Lima e Silva, R.; Shen, J.; Hackett, S. F.; Kachi, S.; Akiyama, H.; Kiuchi, K.; Yokoi, K.; Hatara, M. C.; Lauer, T.; Aslam, S.; Gong, Y. Y.; Xiao, W. H.; Khu, N. H.; Thut, C.; Campochiaro, P. A., The SDF-1/CXCR4 ligand/receptor pair is an important contributor to several types of ocular neovascularization. FASEB journal: official publication of the Federation of American Societies for Experimental Biology 2007, 21 (12), 3219-30.
37. Jarrett, S. G.; Boulton, M. E., Consequences of oxidative stress in age-related macular degeneration. Mol Aspects Med 2012, 33 (4), 399-417.
38. (a) Wang, A. L.; Lukas, T. J.; Yuan, M.; Neufeld, A. H., Increased mitochondrial DNA damage and down-regulation of DNA repair enzymes in aged rodent retinal pigment epithelium and choroid. Molecular vision 2008, 14, 644-51; (b) Boulton, M.; Rozanowska, M.; Rozanowski, B., Retinal photodamage. J Photochem Photobiol B 2001, 64 (2-3), 144-61; (c) Kopitz, J.; Holz, F. G.; Kaemmerer, E.; Schutt, F., Lipids and lipid peroxidation products in the pathogenesis of age-related macular degeneration. Biochimie 2004, 86 (11), 825-31.
39. Doyle, S. L.; Campbell, M.; Ozaki, E.; Salomon, R. G.; Mori, A.; Kenna, P. F.; Farrar, G. J.; Kiang, A. S.; Humphries, M. M.; Lavelle, E. C.; O'Neill, L. A. J.; Hollyfield, J. G.; Humphries, P., NLRP3 has a protective role in age-related macular degeneration through the induction of IL-18 by drusen components. Nature medicine 2012, 18 (5), 791-U191.
40. (a) Cai, X.; Seal, S.; McGinnis, J. F., Sustained inhibition of neovascularization in vldlr−/− mice following intravitreal injection of cerium oxide nanoparticles and the role of the ASK1-P38/JNK-NF-kappaB pathway. Biomaterials 2014, 35 (1), 249-58; (b) Imamura, Y.; Noda, S.; Hashizume, K.; Shinoda, K.; Yamaguchi, M.; Uchiyama, S.; Shimizu, T.; Mizushima, Y.; Shirasawa, T.; Tsubota, K., Drusen, choroidal neovascularization, and retinal pigment epithelium dysfunction in SOD1-deficient mice: A model of age-related macular degeneration. P Natl Acad Sci USA 2006, 103 (30), 11282-11287; (c) Zhao, Z.; Chen, Y.; Wang, J.; Sternberg, P.; Freeman, M. L.; Grossniklaus, H. E.; Cai, J., Age-related retinopathy in NRF2-deficient mice. Plos One 2011, 6 (4), e19456; (d) Justilien, V.; Pang, J. J.; Renganathan, K.; Zhan, X.; Crabb, J. W.; Kim, S. R.; Sparrow, J. R.; Hauswirth, W. W.; Lewin, A. S., SOD2 knockdown mouse model of early AMD. Invest Ophthalmol Vis Sci 2007, 48 (10), 4407-20.
41. (a) Dong, A.; Xie, B.; Shen, J.; Yoshida, T.; Yokoi, K.; Hackett, S. F.; Campochiaro, P. A., Oxidative stress promotes ocular neovascularization. J Cell Physiol 2009, 219 (3), 544-52; (b) Dorrell, M. I.; Aguilar, E.; Jacobson, R.; Yanes, O.; Gariano, R.; Heckenlively, J.; Banin, E.; Ramirez, G. A.; Gasmi, M.; Bird, A.; Siuzdak, G.; Friedlander, M., Antioxidant or neurotrophic factor treatment preserves function in a mouse model of neovascularization-associated oxidative stress. The Journal of clinical investigation 2009, 119 (3), 611-23.
42. Kwak, N.; Okamoto, N.; Wood, J. M.; Campochiaro, P. A., VEGF is major stimulator in model of choroidal neovascularization. Invest Ophthalmol Vis Sci 2000, 41 (10), 3158-64.
43. Ushio-Fukai, M., VEGF signaling through NADPH oxidase-derived ROS. Antioxid Redox Signal 2007, 9 (6), 731-9.
44. (a) Yoo, H. S.; Lee, J. E.; Chung, H.; Kwon, I. C.; Jeong, S. Y., Self-assembled nanoparticles containing hydrophobically modified glycol chitosan for gene delivery. Journal of controlled release: official journal of the Controlled Release Society 2005, 103 (1), 235-43; (b) Na, J. H.; Lee, S. Y.; Lee, S.; Koo, H.; Min, K. H.; Jeong, S. Y.; Yuk, S. H.; Kim, K.; Kwon, I. C., Effect of the stability and deformability of self-assembled glycol chitosan nanoparticles on tumor-targeting efficiency. Journal of controlled release: official journal of the Controlled Release Society 2012, 163 (1), 2-9; (c) Kim, J. H.; Kim, Y. S.; Park, K.; Lee, S.; Nam, H. Y.; Min, K. H.; Jo, H. G.; Park, J. H.; Choi, K.; Jeong, S. Y.; Park, R. W.; Kim, I. S.; Kim, K.; Kwon, I. C., Antitumor efficacy of cisplatin-loaded glycol chitosan nanoparticles in tumor-bearing mice. Journal of controlled release: official journal of the Controlled Release Society 2008, 127 (1), 41-9; (d) Kim, J. H.; Kim, Y. S.; Park, K.; Kang, E.; Lee, S.; Nam, H. Y.; Kim, K.; Park, J. H.; Chi, D. Y.; Park, R. W.; Kim, I. S.; Choi, K.; Chan Kwon, I., Self-assembled glycol chitosan nanoparticles for the sustained and prolonged delivery of antiangiogenic small peptide drugs in cancer therapy. Biomaterials 2008, 29 (12), 1920-30.
45. Koo, H.; Moon, H.; Han, H.; Na, J. H.; Huh, M. S.; Park, J. H.; Woo, S. J.; Park, K. H.; Kwon, I. C.; Kim, K.; Kim, H., The movement of self-assembled amphiphilic polymeric nanoparticles in the vitreous and retina after intravitreal injection. Biomaterials 2012, 33 (12), 3485-93.
46. Wan, A.; Xu, Q.; Sun, Y.; Li, H., Antioxidant activity of high molecular weight chitosan and N,O-quaternized chitosans. J Agric Food Chem 2013, 61 (28), 6921-8.
47. (a) Kliffen, M.; Sharma, H. S.; Mooy, C. M.; Kerkvliet, S.; de Jong, P. T., Increased expression of angiogenic growth factors in age-related maculopathy. The British journal of ophthalmology 1997, 81 (2), 154-62; (b) Yi, X.; Ogata, N.; Komada, M.; Yamamoto, C.; Takahashi, K.; Omori, K.; Uyama, M., Vascular endothelial growth factor expression in choroidal neovascularization in rats. Graefes Arch Clin Exp Ophthalmol 1997, 235 (5), 313-9; (c) Ryan, S. J., Subretinal neovascularization. Natural history of an experimental model. Arch Ophthalmol 1982, 100 (11), 1804-9.
48. Chetram, M. A.; Hinton, C. V., ROS-mediated regulation of CXCR4 in cancer. Front Biol (Beijing) 2013, 8 (3).
49. Hong, X.; Jiang, F.; Kalkanis, S. N.; Zhang, Z. G.; Zhang, X. P.; DeCarvalho, A. C.; Katakowski, M.; Bobbitt, K.; Mikkelsen, T.; Chopp, M., SDF-1 and CXCR4 are up-regulated by VEGF and contribute to glioma cell invasion. Cancer letters 2006, 236 (1), 39-45.
50. Mitra, R. N.; Merwin, M. J.; Han, Z.; Conley, S. M.; Al-Ubaidi, M. R.; Naash, M. I., Yttrium oxide nanoparticles prevent photoreceptor death in a light-damage model of retinal degeneration. Free radical biology & medicine 2014.
51. (a) Mitra, R. N.; Nichols, C. A.; Guo, J.; Makkia, R.; Cooper, M. J.; Naash, M. I.; Han, Z., Nanoparticle-mediated miR200-b delivery for the treatment of diabetic retinopathy. Journal of controlled release: official journal of the Controlled Release Society 2016, 236, 31-7; (b) Arnaoutova, I.; Kleinman, H. K., In vitro angiogenesis: endothelial cell tube formation on gelled basement membrane extract. Nature protocols 2010, 5 (4), 628-35.
52. Liang, C. C.; Park, A. Y.; Guan, J. L., In vitro scratch assay: a convenient and inexpensive method for analysis of cell migration in vitro. Nature protocols 2007, 2 (2), 329-33.
53. Mitra, R. N.; Han, Z.; Merwin, M.; Al Taai, M.; Conley, S. M.; Naash, M. I., Synthesis and characterization of glycol chitosan DNA nanoparticles for retinal gene delivery. ChemMedChem 2014, 9 (1), 189-96.

### Example 2

A 0.2 ml solution of 0.7 M CeCl₃, 7H₂O (Sigma, 99%) was added to 2.5 ml of 2% (w/v) of glycol chitosan (Sigma, degree of polymerization ≥400) solution under constant stirring at room temperature for 10 min. 0.1 ml of concentrated ammonium hydroxide (Sigma, 28-30%) was slowly added to the mixture. After complete addition of the ammonium hydroxide, an equal volume of water was added to the solution mixture and stirred for 24 hrs at room temperature. The color of the solution changed from colorless to yellow the next day, indicating the formation of nanoceria particles stabilized with GC. Thereafter, solution was centrifuged at 14K rpm for 20 min at room temperature to precipitate out any aggregation. The supernatant was collected to avoid any traces of aggregation. This supernatant was then dialyzed using slide-A-lyzer dialysis cassettes (Thermo Fisher Scientific, 10 kDa MWCO cut off) for 2 days at room temperature against nanopure water. After dialysis, the solution was neutralized using acetic acid (Sigma, >99%) to physiological pH 7.4. The color of the solution remained yellowish. The nanoceria solution was then lyophilized and the yellowish solid was stored at −20° C. for further use. On further use, the lyophilized material was just dissolved in water and warmed at 75° C. for 10 mins to get the clear and transparent solution of water soluble GC capped nanoceria.

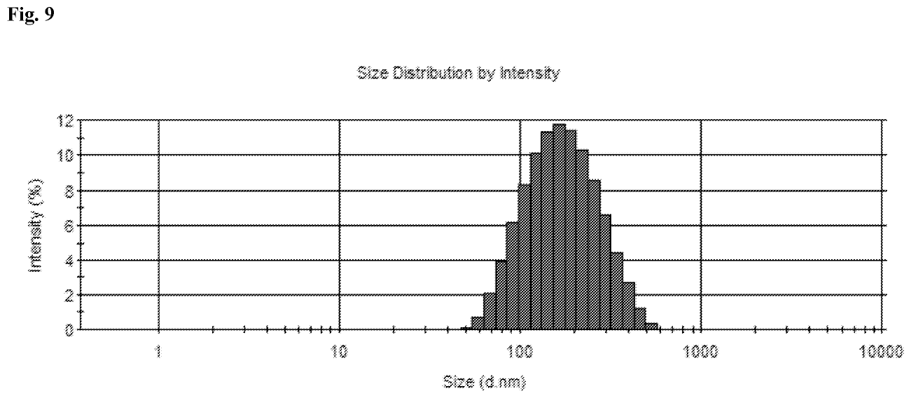

The ceria nanoparticles were first characterized right after synthesis by H₂O₂ addition. Addition of H₂O₂ leads to the change in color of the mixed valence Ce⁺³/Ce⁺⁴ (colorless) solution to yellowish solution. This yellowish solution returns back to its original color after 7-10 days at room temperature. High-resolution TEM (HR-TEM) images of the GC capped ceria nanoparticle solution were taken and it was found that the nanoparticles were monodispersed and non-agglomerated 3-5 nm ceria nanoparticles stabilized by the GC matrix. HR-TEM images indicated the atomic spacing of 0.314 nm that is characteristic of cubic phase with fluorite structure of ceria nanoparticles. The selected area electron diffraction (SAED) pattern revealed a cubic fluorite structure of ceria nanoparticles with the most intense line corresponding to (111) that is a characteristic to 0.312 nm lattice spacing. The energy-dispersive X-ray spectroscopy (EDX) pattern confirmed the presence of cerium. Dynamic light scattering (DLS) measurements of the GC capped ceria NPs (GCC NP) were carried out and revealed average hydrodynamic diameter of the particles was ˜140 nm (PDI=0.258, FIG. 9), that might be due to the formation of a large GC matrix in solution as also found in TEM. The zeta potential of the water soluble particles was ˜+30 mV. The materials didn't change any shape or size even after 1 year of storage at −20° C. The NPs (5 different concentrations) were then incubated overnight with H₂O₂-treated ARPE19 cells and DCF assay was carried out with 10 μM DCFH-DA and the fluorescence of DCF was determined with excitation 485 nm and emission 530 nm. The results revealed that the NPs could scavenge ROS with a significant amount (n=5, p<0.0001) compared to the uninjected and mock injected controls. This nanoparticle (2 ug/ul) was injected into the laser treated eye of C57Bl6/j mice (adult) at the intravitreal space (2 ul) after 2 days of laser injury. It was found that after 2 months of post injection (PI-2m), a significant amount of healing of the injured compared with the uninjected and saline injected controls. Eyes has also been collected and the vascular endothelial growth factor (VEGF) protein expression has been measured using western blot. It was found that VEGF expression level has been significantly reduced in NP treated eye samples compared with the controls.

### Example 3

We have developed glycol chitosan-coated cerium nanoparticles (GCCNPs) that are water soluble. We have demonstrated that these GCCNPs serve as an auto-generative antioxidant to attenuate pathological damages in models of age-related macular degeneration. GCCNPs belong to the family of cerium oxide nanoparticles (CNPs).

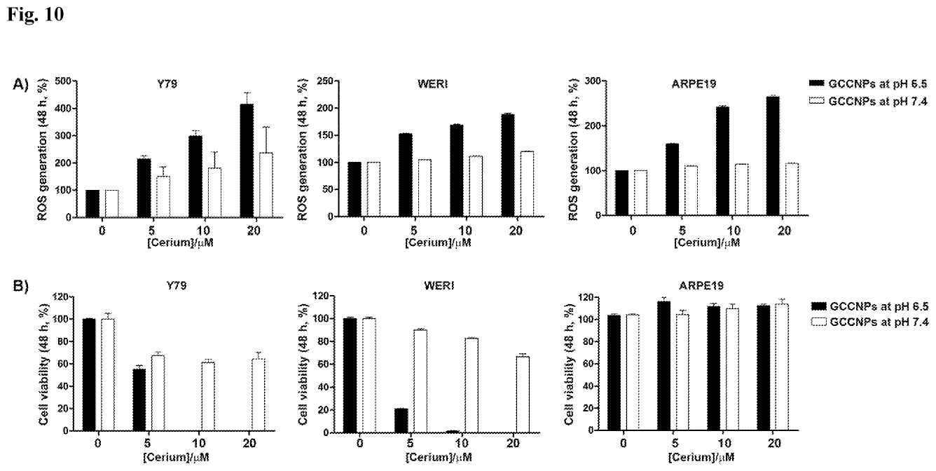

Data from long-term (4 cycle) auto-regenerative redox activity and reactive free radical generating capacity (at pH 6.5) of GCCNPs (FIGS. 10A and 10B) is provided herein. For the auto-regenerative redox activity experiment, water was used as media for the NPs and GCCNPs, and H₂O₂ addition induces ROS in the medium.

The pH-responsiveness is one of the most attractive and frequently applied stimuli to treat tumor conditions. It was observed that the tumor extracellular environment is more acidic (pH=6.5) than the normal and blood (pH=7.4). In addition, the pH of endosomes and lysosomes is also lower (pH=5.0-5.5). Based on this phenomena and naturally attractive pH stimulant, several drug delivery systems have been developed, including polymeric supramolecular systems (micelles, gels etc.) and pH triggered therapeutic/diagnostics releases. However, most of these systems were focused or explored significantly on the acidic endo/lysosomal conditions. The exploration of novel carriers that can be functionalized in tumor extracellular pH condition has become an important tool for tumor targeted drug deliveries. In this context, we used GCCNPs as a base vector for targeted drug deliveries for the tumor. As the pH of tumor microenvironment is acidic, GCCNPs behave like an oxidant and exerted cytotoxicity to the tumor cells (e.g., Y79 and WERI), but not the normal cells (ARPE19). Our data showed that the GGCNPs can produce ROS in a significant quantity in tumor cells under an acidic extracellular environment (pH=6.5) in a dose dependent manner (FIG. 10A). The results also demonstrated antitumor activities (cell viability) in a tumor microenvironment (acidic condition, pH=6.5) in a dose dependent manner (FIG. 10B). Selectively targeting cancer cells while protecting normal cells is another highlight of the GCCNPs as a robust intelligent drug delivery system for tumor suppression. While not wishing to be bound to any particular theory, the ARPE19 cells are believed to have remained viable at pH 6.5 even though the GCCNPs generated ROS since GCCNP have an intrinsic oxidase activity at acidic pH and the pH of the tumor cells (e.g., Y79 and WERI cells) is more acidic than that of normal cells (e.g., ARPE19). Plus, there is a strong endogenous antioxidant system in normal cells to protect them from oxidative stress induced apoptosis.

### Methods:

### Auto-Regenerative Property:

We took 1 mg/ml of naked ceria NPs (1^{st} tube), and GCCNPs (2^{nd} and 3^{rd} tubes) in glass vials separately. We added 1 mM of H₂O₂ to 1^{st} and 3^{rd} vial, changing the color of the samples to dark yellow, indicating the oxidation of Ce³⁺ to Ce⁴⁺. By day 7, the color of the samples reverted back to colorless, indicating the further reduction of Ce⁴⁺ and regeneration of Ce³⁺. We repeated the oxidation process with 1 mM of H₂O₂ to 4 cycles now, and it continued to replicate. This experiment concludes that our GCCNPs were reusable and had the abilities to continue to scavenge free radicals. Also, through the experiments, the naked nanoceria (1^{st} tube) remained insoluble, whereas the GCCNPs showed a clear solution.

### Cell Culture:

A human retinal pigment epithelial cell line, ARPE-19, was adherently cultured in DMEM/F12+GlutaMAX™-I media supplemented with 10% fetal bovine serum (FBS) and a 1% antibiotic-antimycotic solution (100×). The human retinoblastoma WERI-Rb-1 and Y79 cell lines were suspension cells and maintained in RPMI medium 1640 (1×) with 1% antibiotic-antimycotic solution containing 10% FBS and 20% FBS, respectively. All cells were cultured at 37° C. in an incubator with 5% CO₂. When subculturing, the adherent cells were enzymatically detached from the surface of the T-25 cell culture flasks using trypsin-EDTA (0.05%, Gibco Life Technology). The suspension cells were collected by centrifugation at 100×g for 3 min.

### pH Adjustment:

To determine the effects of pH value on the viability and ROS generation of GCCNPs, 4 of the pH values (6.5, 7.0, 7.4, and 8.0) arranging from acidity to base values were set up. pH 6.5 is the approximate pH value of the tumor microenvironment and pH 7.0 represents the neutral pH value. pH 7.4 represents the physical pH value for regular cell growth and pH 8 is a closer pH value of the commercial cell culture media used in this article. To reduce the pH values to less than 8.0, 1 M or 10 M acetic acid (Sigma-Aldrich, USA) was used according to the need. An aqueous solution of 1 M or 10 M sodium hydroxide (Sigma-Aldrich, USA) was used to increase the pH value. The pH value of the solution was measured by a pH meter.

### Cell Viability:

Both ARPE 19 (4×10³ cells/well) and RB cells (Y79 and WERI; 2.5×10⁴ cells/well) were seeded in a 96-well plate and incubated overnight. Cells were exposed to different concentrations of drugs in different pH media for 48 h. After removing the culture supernatant, 100 μl of fresh media were added to each well. For ARPE cells, 10 μl of MTT (5 mg/ml solution in 1× DPBS) were added for an additional 3.5 h. The supernatant containing media and MTT was then discarded, and 150 μl of DMSO was added to each well to dissolve the purple formazan crystals. The optical density at 570 nm (OD570) of each well was determined using a plate reader. For human RB cells, 10 μl of WST-1 reagent was added per well for another 2 h. The optical density at 450 nm (OD450) of each well was measured with the reference wavelength of 690 nm. As the used RB cells do not attach to the substratum, centrifugation with 2000 rpm for 10 min was required when changing the medium.

### Reactive Oxygen Species (ROS) Measurements:

The generation of intracellular ROS was measured using a cell-permeable non-fluorescent probe 2′,7′-dichlorofluorescin diacetate (DCFH-DA, #D6883) probe which would be de-esterified intracellularly and oxidized to highly fluorescent 2′,7′-dichlorofluorescin (DCF) upon oxidation. Cells were planted in 96-well plates and cultured for 24 h. Next day, cells were treated with different concentrations of GCCNPs at pH 6.5 and pH 7.4 for 48 h. Cells were washed with warm Hank's Balanced Salt Solution (HBSS) twice and trypsinized for 5 min with 20 μl 0.5% trypsin solution. After that, 90 μL of 25 μM DCFH-DA was added to each well. Finally, 100 μl of cell and dye suspension were transferred into white 96-well plates and incubated at 37° C. for 45 min. The fluorescent intensity was determined using a SpectraMax M3 (mt05412) spectrophotometer at 485/530 nm.

### Example 4

Cerium oxide nanoparticles (nanoceria) have unique redox activity in that they possess antioxidant activity at physiological pH, but an intrinsic oxidase activity at acidic pH. Therefore, nanoceria can be used as a base vector for anti-tumor therapy since it can inhibit cancer cells without damaging fellow normal tissues. We have designed a novel antitumor delivery system using a chemotherapy drug and tumor target molecules covalently linked to nanoceria. The goal of this project was to develop nanoceria-assisted combination therapies for tumor-directed treatment of retinoblastoma.

Methods: The compound GCCNP-DOX-AMD070 was constructed, which contains a chemotherapeutic drug (i.e., Doxorubincin, DOX) and a tumor selective targeting reagent (i.e., AMD070, a CXCR4 antagonist) via surface chemistry of our developed water-soluble nanoceria (GCCNP). Dynamic light scattering (DLS) measurements were performed to confirm the successful construction of the compound. Quantitative assessment of in vitro intracellular reactive oxygen species (ROS) production, cell viability, and CXCR4 (a cancer progression marker) expression were performed by DCF, MTT, and Western blot assays at different pH levels in human tumor cell lines (WERI-Bb-1, Y79, A549, and B16F10) and normal human ARPE-19 cells.

Results: We observed strong CXCR4 expressions in all the tumor cell lines but barely in normal ARPE-19 cells. We detected that the GCCNPs increase the production of ROS and reduce cell viability at pH 6.5 but not at pH 7.4 in all the cell lines that we tested. We found that our combo system markedly suppressed Y79 CXCR4 expression at pH 6.5 compared with that treated with DOX only. Interestingly, we did not find the same changes when pH was 7.4, indicating the pH-dependent antitumor behavior of GCCNP-DOX-AMD070.

The results demonstrate that GCCNP-DOX-AMD070 is capable of specifically and selectively targeting tumor cells under tumor microenvironment (acidic conditions), but does not produce ROS or produces a minimal amount of ROS under normal physiological pH conditions, thus reducing the problems of off-target. The design of nanoceria combo in this application can be custom-built and functionalized to target different diseases by binding the surface of nanoparticles with a cell-specific targeting molecule and therapeutic specificity.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

1. A water-soluble nanoceria comprising a cerium oxide nanoparticle and glycol chitosan.

2. The water-soluble nanoceria of claim 1, wherein the cerium oxide nanoparticle is prepared from a cerium salt.

3. (canceled)

4. The water-soluble nanoceria of claim 1, wherein the cerium oxide nanoparticle is present in an amount of about 10% to about 30% by weight of the water-soluble nanoccria and the glycol chitosan is present in an amount of about 60% to about 80% by weight of the water-soluble nanoceria.

5. The water-soluble nanoceria of claim 1, wherein the glycol chitosan coats at least a portion of an exterior surface of the cerium oxide nanoparticle.

6. The water-soluble nanoceria of claim 1, wherein at least one therapeutic agent and/or targeting agent is attached to a surface of the water-soluble nanoceria.

7. The water-soluble nanoceria of claim 1, wherein the cerium oxide nanoparticle has a diameter in a range of about 2 nm to about 6 nm.

8. The water-soluble nanoceria of claim 1, wherein the cerium oxide nanoparticle has a crystalline structure.

9. The water-soluble nanoceria of claim 1, wherein the cerium oxide nanoparticle is monodisperse.

10. (canceled)

11. The water-soluble nanoceria of claim 1, wherein the cerium oxide nanoparticle has a cubic fluorite structure.

12. The water-soluble nanoceria of claim 1, wherein the water-soluble nanoceria have a hydrodynamic diameter in a range of about 100 nm to about 200 nm.

13. The water-soluble nanoceria of claim 1, wherein the water-soluble nanoceria have a zeta potential in a range of about +10 to about +40.

14. The water-soluble nanoceria of claim 1, wherein the water-soluble nanoceria scavenge free radicals and/or quench free radicals.

15. The water-soluble nanoceria of claim 1, wherein the water-soluble nanoceria are positively charged.

16. The water-soluble nanoceria of claim 1, wherein the water-soluble nanoceria have a water solubility in a range of about 0.0001 mg/ml to about 25 mg/ml.

17-19. (canceled)

20. A composition comprising the water-soluble nanoceria of claim 1.

21. The composition of claim 20, further comprising water.

22. The composition of claim 21, wherein the composition is stable at room temperature for about a year.

23. (canceled)

24. A method of preparing a water-soluble nanoceria, the method comprising:
combining a cerium salt and glycol chitosan to form a mixture; and
adding a base to the mixture to form a solution, thereby preparing the water-soluble nanoceria.

25.-34. (canceled)

35. A method of treating an oxidative stress-related disorder, cancer and/or neurodegenerative disorder in a subject, the method comprising:
administering the water-soluble nanoceria of claim 1 to the subject, thereby treating the oxidative stress-related disorder, cancer, and/or neurodegenerative disorder in the subject.

36. (canceled)

37. A method of decreasing reactive oxygen species in a subject, the method comprising:
administering the water-soluble nanoceria of claim 1 to the subject, thereby decreasing reactive oxygen species in the subject.

38. (canceled)
